(19) United States
(12) Patent Application Publication
(10) Pub. No.: US 2022/0370306 A1
(43) Pub. Date: Nov. 24, 2022

(54) SILICONE DROPLETS

(71) Applicant: FORMULAE 8 PTY LTD, New South Wales (AU)

(72) Inventor: Danilo Lambino, New South Wales (AU)

(21) Appl. No.: 17/763,073

(22) PCT Filed: Sep. 22, 2020
(86) PCT No.: PCT/AU2020/051000

§ 371 Date: Mar. 23, 2022

(30) Foreign Application Priority Data
Sep. 25, 2019 (AU) 2019903575

(51) Int. Cl. A61K 8/04; A61K 8/895; A61K 8/891; A61Q 19/02; A61Q 17/04

(52) U.S. Cl. CPC A61K 8/04; A61K 8/895; A61K 8/891; A61Q 19/02; A61Q 17/04; A61K 2800/48; A61K 2800/33

(57) ABSTRACT

The present invention relates generally to compositions comprising silicone droplets dispersed in an aqueous phase and in particular to cosmetic compositions comprising silicone droplets dispersed in an aqueous phase.

The present invention relates generally to compositions comprising silicone droplets dispersed in an aqueous phase and in particular to cosmetic compositions comprising silicone droplets dispersed in an aqueous phase.

### FIELD OF THE DISCLOSURE

The present invention relates generally to compositions comprising silicone droplets dispersed in an aqueous phase and in particular to cosmetic compositions comprising silicone droplets dispersed in an aqueous phase.

### BACKGROUND OF THE DISCLOSURE

Any discussion of the prior art throughout this specification should in no way be considered as an admission that such prior art is widely known or forms part of the common general knowledge in the field.

Formulations comprising dispersed droplets are widely used in the cosmetics industry. The droplets may be functional or aesthetic.

However, many known formulations comprising droplets are visually or texturally unappealing. For example, certain formulations comprise high concentrations of surfactants (which must be included to produce macro or microemulsions) and have a sticky texture. Moreover, the droplets are often very small, rendering the formulation opaque, and may also be unsuitable for carrying actives that are either incompatible with surfactants or that are susceptible to hydrolysis.

In an effort to produce more visually appealing droplets, researchers have developed droplets in the form of alginate capsules. However, these capsules are not suitable for incorporating lipophilic materials, and they are typically opaque. In many cases, the techniques used to produce the droplets are costly and require complex machinery.

In this context, there is a need for new compositions comprising dispersed droplets.

### SUMMARY OF THE DISCLOSURE

In a first aspect, the present disclosure provides a composition comprising silicone droplets dispersed in an aqueous phase.

In some examples, the aqueous phase comprises water and a thickening agent.

The silicone droplets may be formed from a silicone fluid. The silicone droplets may be formed from a silicone fluid and a silicone crosslinked polymer. The silicone droplets may be formed from a silicone fluid and a silicone gum. The silicone droplets may be formed from a silicone fluid, a silicone crosslinked polymer and a silicone gum.

In some examples, the composition comprises substantially no surfactant.

In some examples, the thickening agent is a gelling agent.

The aqueous phase may further comprise a humectant. The aqueous phase may further comprise a chelating agent. The aqueous phase may further comprise a preservative. The aqueous phase may further comprise a colourant.

In some examples, the silicone droplets comprise dimethicone/vinyl dimethicone crosspolymer.

In some examples, the silicone droplets comprise dimethicone.

In some examples, the silicone droplets comprise a film former.

In some examples, the silicone droplets comprise a lipophilic emollient other than silicone.

In some examples, the silicone droplets comprise a lipophilic active agent.

In some examples, the composition comprises the silicone droplets at a concentration of between about 1% and 50% (w/w).

In some examples, the composition is a cosmetic composition suitable for topical application to human skin or hair.

In a second aspect, the present disclosure provides a cosmetic composition comprising:

an aqueous phase comprising water and a thickening agent; and

a silicone gel,

wherein the silicone gel forms silicone droplets dispersed in the aqueous phase when the composition is agitated.

In some examples, the composition comprises substantially no surfactant.

In some examples, the silicone gel is formed from a silicone fluid.

In some examples, the silicone gel is formed from a silicone fluid and a silicone crosslinked polymer.

In some examples, the silicone gel is formed from a silicone fluid and a silicone gum.

In some examples, the silicone gel is formed from a silicone fluid, a silicone crosslinked polymer and a silicone gum.

In some examples, the thickening agent is a gelling agent.

The aqueous phase may further comprise a humectant. The aqueous phase may further comprise a chelating agent. The aqueous phase may further comprise a preservative. The aqueous phase may further comprise a colourant.

In some examples, the silicone gel comprises dimethicone/vinyl dimethicone crosspolymer.

In some examples, the silicone gel comprises dimethicone.

In some examples, the silicone gel comprises a film former.

In some examples, the silicone gel comprises a lipophilic emollient other than silicone.

In some examples, the silicone gel comprises a lipophilic active agent.

In some examples, the composition comprises the silicone gel at a concentration of between about 1% and 50% (w/w).

In some examples, the silicone gel forms silicone droplets dispersed in the aqueous phase when the composition is shaken or stirred.

The silicone droplets of the first and second aspects may be clear or translucent.

In a third aspect, the present disclosure provides a method of producing a composition comprising silicone droplets dispersed in an aqueous phase, the method comprising:

adding a silicone gel to an aqueous phase, wherein the aqueous phase comprises water and a thickening agent; and

agitating the silicone gel and the aqueous phase such that the silicone gel forms silicone droplets dispersed in the aqueous phase.

In a fourth aspect, the present disclosure provides a method of producing a composition comprising silicone droplets dispersed in an aqueous phase, the method comprising:

stirring an aqueous phase comprising water and a thickening agent; and

adding a silicone gel to the aqueous phase as it is being stirred so as to form silicone droplets dispersed in the aqueous phase.

In some examples, the stirring is performed at a rate of between about 100 rpm and 500 rpm.

In some examples, the composition comprises substantially no surfactant.

In some examples, the silicone gel is formed from a silicone fluid.

In some examples, the method further comprises mixing a silicone fluid and a silicone crosslinked polymer to thereby form the silicone gel.

In some examples, the method further comprises mixing a silicone fluid and a silicone gum to thereby form the silicone gel.

In some examples, the method further comprises mixing a silicone fluid, a silicone crosslinked polymer and a silicone gum to thereby form the silicone gel.

In some examples, the method further comprises mixing a silicone fluid and one or more of a film former, a colourant, a lipophilic emollient other than silicone and a lipophilic active agent to thereby form the silicone gel.

In some examples, the method further comprises mixing a silicone fluid, a silicone crosslinked polymer and one or more of a film former, a colourant, a lipophilic emollient other than silicone and a lipophilic active agent to thereby form the silicone gel.

In some examples, the method further comprises mixing a silicone fluid, a silicone gum and one or more of a film former, a colourant, a lipophilic emollient other than silicone and a lipophilic active agent to thereby form the silicone gel.

In some examples, the method further comprises mixing a silicone fluid, a silicone crosslinked polymer, a silicone gum and one or more of a film former, a colourant, a lipophilic emollient other than silicone and a lipophilic active agent to thereby form the silicone gel.

In some examples, the silicone fluid is dimethicone.

In some examples, the silicone droplets are formed from dimethicone/vinyl dimethicone crosspolymer.

In some examples, the method further comprises mixing water and a thickening agent to thereby produce the aqueous phase.

In some examples, the thickening agent is a gelling agent.

In some examples, the method further comprises mixing water, a thickening agent and one or more of a humectant, a chelating agent, a preservative and a colourant to thereby produce the aqueous phase.

In a fifth aspect, the present disclosure provides a composition when produced by the method of the third aspect or the fourth aspect.

### BRIEF DESCRIPTION OF THE DRAWINGS

FIG. 1. Compositions comprising silicone droplets dispersed in an aqueous phase.

FIG. 2. A composition comprising opaque silicone droplets dispersed in an aqueous phase.

FIG. 3. A composition comprising pearlised silicone droplets dispersed in an aqueous phase.

FIG. 4. A composition comprising sparkling silicone droplets in the form of rods dispersed in an aqueous phase.

FIG. 5. A composition comprising pearlised silicone droplets in the form of strings dispersed in an aqueous phase.

FIG. 6. A composition comprising pearlised silicone droplets in the form of strings dispersed in an aqueous phase.

FIG. 7. Compositions comprising red silicone droplets dispersed in an aqueous phase.

FIG. 8. A composition comprising silicone droplets including UV filters dispersed in an aqueous phase.

FIG. 9. A composition comprising silicone droplets including UV filters dispersed in an aqueous phase.

FIG. 10. A composition comprising silicone droplets dispersed in an aqueous phase.

FIG. 11. A composition comprising a coloured silicone gel and an aqueous phase (left), and a composition comprising coloured silicone droplets dispersed in an aqueous phase.

FIG. 12. A composition comprising clear silicone droplets dispersed in an aqueous phase.

FIG. 13. A composition comprising silicone droplets dispersed in an aqueous phase.

FIG. 14. A composition comprising silicone droplets dispersed in an aqueous phase.

FIG. 15. Compositions comprising silicone droplets dispersed in an aqueous phase.

FIG. 16. A composition comprising silicone droplets dispersed in an aqueous phase.

FIG. 17. Silicone droplets of FIG. 3 at a magnification of 4×.

FIG. 18. Silicone droplets of FIG. 12 at a magnification of 10×.

### DETAILED DESCRIPTION

### Definitions

The articles “a” and “an” are used herein to refer to one or to more than one (i.e. to at least one) of the grammatical object of the article.

The term “about” is understood to refer to a range of +1-10%, preferably +1-5% or +1-1% or, more preferably, +1-0.1%.

Throughout this specification, unless the context requires otherwise, the word “comprise”, or variations such as “comprises” or “comprising” will be understood to imply the inclusion of a stated element, integer or step, or group of elements, integers or steps, but not the exclusion of any other element, integer or step, or group of elements, integers or steps. Thus, in the context of this specification, the term “comprising” means “including principally, but not necessarily solely”.

A “cosmetic” composition is one that is applied, or intended to be applied, to part of a body, typically a human body including, inter alia, hair, skin or nails, for cleansing, beautifying, promoting attractiveness, altering appearance or any combination thereof.

The term “substantially no” as used in reference to surfactant content means that surfactants constitute less than about 3% (w/w), such as less than about 2% (w/w), or less than about 1% (w/w), preferably less than about 0.5% (w/w), or less than about 0.1% (w/w), or less than about 0.05% (w/w), or less than about 0.01% (w/w).

### Compositions

The present disclosure provides compositions comprising silicone droplets dispersed in an aqueous phase. The present disclosure also provides compositions comprising an aqueous phase and a silicone gel, wherein the silicone gel forms silicone droplets dispersed in the aqueous phase when the composition is agitated. The aqueous phase preferably comprises water and a thickening agent. The thickening agent may be a naturally-derived thickening agent such as xanthan gum, dehydroxanthan gum, sclerotium gum, gellan gum, pullulan, succinoglycan, Caesalpinia spinosa (Tara) gum, Acacia senegal gum, Cyamopsis tetragonoloba (Guar) gum or its derivatives hydroxypropyl guar and carboxymethyl hydroxypropyl guar, Amorphophallus konjac root gum, pectin, locust bean gum, tragacanth gum, cellulose or its derivatives ethyl cellulose, methyl cellulose, hydroxyethyl cellulose and hydroxymethyl cellulose, carrageenan, agarose, agar, algin, calcium alginate, gelatin, magnesium aluminium silicate, bentonite, hectorite, attapulgite, laponite, sepiolite or silicate. In some examples, the thickening agent is a biofermentation product, a plant extract, a seaweed extract, an algae extract or a mineral compound. In some examples, the thickening agent is a polymeric thickening agent such as sodium polyacrylate, sodium polyacrylate starch, carbomer, acrylates/C10-30 alkyl acrylates crosspolymer or polyacrylate crosspolymer-6, ammonium acryloyldimethyltaurate/VP copolymer, PVP, acrylic acid/VP crosspolymer, acrylates/C10-30 alkyl methacrylate copolymer, polyacrylate-1 crosspolymer, acrylates copolymer, acrylates crosspolymer-4, acrylates crosspolymer-3, sodium carboxymethyl starch, polyacrylate-14 or maltodextrin VP copolymer. Those skilled in the art will understand that other thickening agents may be suitably used in the compositions of the present disclosure. In preferred examples, the thickening agent is a gelling agent.

The thickening agent may be present in the composition at a concentration of between about 0.1% and 5% (w/w), such as between about 0.2% and 4% (w/w), or between about 0.3% and 4% (w/w), or between about 0.4% and 3% (w/w), or between about 0.5% and 2% (w/w), or between about 0.6% and 1.5% (w/w), or between about 0.7% and 1.4% (w/w), or between about 0.8% and 1.3% (w/w), or between about 0.9% and 1.2% (w/w), such as about 1% or about 1.1% (w/w). In examples where the aqueous phase comprises more than one thickening agent, these concentrations refer to the combined total of the thickening agents. For example, a composition comprising a thickening agent at a concentration of 1% (w/w) may comprise 0.8% (w/w) xanthan gum and 0.2% (w/w) sclerotium gum. It will be understood that these concentrations are made in reference to the composition inclusive of the aqueous phase and the dispersed silicone droplets.

In certain examples, the aqueous phase has a viscosity of between about 200 and 100,000 cps, such as between about 1,000 and 100,000 cps, or between about 1,100 and 90,000 cps, or between about 1,200 and 80,000 cps, or between about 1,300 and 75,000 cps, or between about 1,400 and 70,000 cps, or between about 1,500 and 65,000 cps, or between about 1,600 and 60,000, or between about 1,700 and 55,000 cps, or between about 1,700 and 50,000 cps, or between about 1,800 and 50,000 cps, or between about 1,900 and 50,000 cps, or between about 2,000 and 50,000 cps at 25° C.

In certain examples, the aqueous phase has a yield stress of between about 1 and 500 Pa, such as between about 5 and 450 Pa, or between about 10 and 400 Pa, or between about 20 and 400 Pa, or between about 30 and 350 Pa, or between about 40 and 350 Pa, or between about 50 and 300 Pa, or between about 60 Pa and 250 Pa at 25° C.

The aqueous phase may further comprise a humectant, a chelating agent, an antioxidant, a pH adjuster, a preservative, a colourant, an opacifier, a cosmetically active agent, a biologically active agent, a skin feel agent, a film forming agent, a perfuming agent or a sunscreen agent. It will be understood that the “a” and “an” in this context may mean “one or more”. For example, an aqueous phase comprising water and a thickening agent may comprise two thickening agents, such as xantham gum and sclerotium gum.

Suitable humectants may include, but are not limited to, glycerine, propylene glycol, pentylene glycol, butylene glycol, propanediol, methyl propanediol, trehalose, sodium PCA, hyaluronic acid, sodium hyaluronate, sodium lactate, amino acids, urea, sugars, xylitol, anhydroxylitol, polyglycerin-3, diglycerin, sorbitol, erythritol, PEG-8, 1,2-hexanediol, polyglutamic acid, hexylene glycol, betaine and inositol.

Suitable chelating agents may include, but are not limited to, ethylenediaminetetraacetic acid (EDTA), disodium EDTA, tetrasodium EDTA, sodium phytate, phytic acid, trisodium ethylenediamine disuccinate, tetrasodium glutamate diacetate, etidronic acid and citric acid.

Suitable pH adjusters may include, but are not limited to, citric acid, sodium citrate, phytic acid, sodium phytate, triethanolamine, sodium hydroxide, potassium hydroxide, tris amino and aminomethyl propanol.

Suitable preservatives may include, but are not limited to, an alcohol, phenoxyethanol, phenethyl alcohol, benzyl alcohol, chlorphenesin, lodopropyl butylcarbamate, methylparaben, propylparaben, ethylparaben, an isothiazolinone, imidazolidinyl urea, DMDM hydantoin, an organic acid such as benzoic acid/sodium benzoate, dehydroacetic acid/sodium dehydroacetate, sorbic acid/potassium sorbate and salicylic acid, ethyl lauroyl arginate HCL, lactoperoxidase and glucose oxidase/glucose.

Suitable colourants may include, but are not limited to, dyes and pigments. Dyes may confer transparent colours and pigments may confer opaque colours. Suitable dyes may include, but are not limited to, FD&C Blue No.1, D&C Red No. 33, FD&C Blue No. 12, FD&C Green No. 13, FD&C Red No. 13, FD&C Red. 33, FD&C Red No. 140, FD&C Yellow No. 15, FD&C Yellow No. 16, D&C Blue No. 14, D&C Blue No. 19; D&C Green No. 15, D&C Green No. 16, D&C Green No. 18, D&C Orange No. 14, D&C Orange No.15, D&C Orange No. 110, D&C Orange No. 111, D&C Orange No. 117, FD&C Red No, 14, D&C Red No. 16, D&C Red No. 17, D&C Red No. 18, D&C Red No. 19, D&C Red No. 117. D&C Red No. 119, D&C Red No. 121, D&C Red No. 122, D&C Red No. 127, D&C Red No. 128, D&C Red No. 130, D&C Red No. 131, D&C Red No. 134, D&C Red No. 139, FD&C Red No. 140, D&C Violet No. 12, D&C Yellow No. 17, Ext. D&C Yellow No. 17, D&C Yellow No. 18, D&C Yellow No. 111, D&C Brown No. 11, Ext. D&C Violet No. 12, D&C Blue No. 16 and D&C Yellow No. 110. Suitable pigments may include, but are not limited to, inorganic pigments, metal oxides or hydroxides, mica, organic pigments, pearlescent pigments, mineral silicates, porous materials, carbons, interference pigments. In some examples, the aqueous phase or the composition is not opaque.

Suitable water-soluble natural dyes may include Melia azadirachta flower extract, Melia azadirachta leaf extract, Lawsonia inermis (Henna) flower/fruit/leaf extract, Ocimum sanctum leaf extract, Ocimum basilicum (Basil) flower/leaf extract, Curcuma longa (Turmeric) root extract, mica, pearl powder (Margarita powder), Aqua (Campo Siddha Neer Ellam Manjal), amino-esters-1, Eclipta prostrata extract, Corallina officinalis extract, Aqua (Campo Siddha Neer Karum Karuppu), betaine, Coccinia indica fruit extract and pearl powder (Campo Siddha Neer Ellam Gopi).

Suitable water-dispersible sunscreens may include, but are not limited to, zinc oxide and titanium dioxide.

Suitable cosmetically or biologically active agents may include, but are not limited to, skin lightening agents, exfoliating agents, antioxidants, peptides, anti-acne agents, anti-inflammatory agents and anti-irritants. Suitable water-soluble skin lightening agents may include, but are not limited to, hydroquinone, azelaic add, butyl hydroxy anisole, gallic acid and its derivatives, kojic add, arbutin, mulberry extract, undecylenoyl phenylalanine, niacinamide and derivatives, vitamin C and derivatives including ascorbyl glucoside, magnesium ascorbyl phosphate and glyceryl ascorbate, soy extract and soy trypsin inhibitors, alpha bisabolol, glucosamine, silvervine extract and plankton extract. Suitable exfoliating agents may include, but are not limited to, beta hydroxyl acids, salicylic acid, alpha hydroxy acids, glycolic acids, fruit acids, polyhydroxy acids, gluconolactone and aldobionic acids. Suitable water-soluble antioxidants may include, but are not limited to, ascorbic acid, glutathione and uric acid, glyceryl ascorbate, carnosine, tea extracts, including green tea extract and olive leaf extract. Suitable peptides may include, but are not limited to, acetyl hexapeptide-8, acetyl heptapeptide-9, palmitoyl tripeptide-1, palmitoyl tripeptide-5, palmitoyl-oligopeptide, palmitoyl tetrapeptide-7 and acetyl octapeptide-3. Suitable anti-acne agents may include, but are not limited to, capryloyl glycine, methylglycine, cinnamon bark extract, zinc PCA, zinc gluconate, zinc lactate, salicylic acid and niacinamide. Suitable anti-irritant or anti-inflammatory agents may include, but are not limited to, allantoin, panthenol, alpha-bisabolol, dipotassium glycyrrhizinate, chamomile extract, aloe vera extract, madecassoside and centella asiatica extract. Suitable water-soluble perfuming agents may include, but are not limited to, floral waters.

In examples where the aqueous phase comprises a humectant, the humectant may be present in the composition at a concentration of between about 1% and 20% (w/w) such as between about 4% and 20% (w/w), or between about 5% and 20% (w/w), or between about 5% and 15% (w/w), or between about 6% and 14% (w/w), or between about 7% and 13% (w/w), or between about 8% and 13% (w/w), or between about 9% and 13% (w/w), or between about 10% and 12% (w/w).

In examples where the aqueous phase comprises a chelating agent, the chelating agent may be present in the composition at a concentration of less than about 1% (w/w), such as less than about 0.5% (w/w), less than about 0.4% (w/w), or less than about 0.3% (w/w), such as about 0.1% (w/w) or about 0.2% (w/w).

In examples where the aqueous phase comprises a preservative, the preservative may be present in the composition at a concentration of between about 0.1% and 5% (w/w), such as between about 0.1% and 4% (w/w), or between about 0.1% and 3% (w/w), or between about 0.1% and 2% (w/w), or between about 0.1% and 1.5% (w/w), or between about 0.1% and 1% (w/w), or between about 0.2% and 1% (w/w), or between about 0.2% and 0.9% (w/w), or between about 0.3% and 0.8% (w/w).

In examples where the aqueous phase comprises a colourant, the colourant may be present in the composition at a concentration of less than about 1% (w/w), such as less than about 0.5% (w/w), or less than about 0.4% (w/w), or less than about 0.3% (w/w), or less than about 0.2% (w/w), or less than about 0.1% (w/w), or less than about 0.05% (w/w), such as about 0.01% (w/w).

In examples where the aqueous phase comprises an anti-irritant or an anti-inflammatory agent, the agent may be present in the composition at a concentration of between about 0.01% and 5% (w/w) such as between about 0.05% and 4% (w/w), or between about 0.1% and 3% (w/w), or between about 0.1% and 2% (w/w), or between about 0.1% and 1% (w/w) such as about 0.1% (w/w) or about 0.5% (w/w).

In examples where the aqueous phase comprises a skin lightening agent, the agent may be present in the composition at a concentration of between about 1% and 10% (w/w) such as between about 1.5% and 8% (w/w), or between about 2% and 7% (w/w), or between about 2% and 6% (w/w), or between about 2% and 5% (w/w), or between about 3% and 5% (w/w).

In examples where the aqueous phase comprises a perfuming agent, the agent may be present in the composition at a concentration of between about 1% and 40% (w/w) such as between about 5% and 35% (w/w), or between about 5% and 30% (w/w), or between about 10% about 30% (w/w), or between about 10% and 25% (w/w) or between about 15% and 25% (w/w).

The silicon droplets of the present disclosure are preferably formed from a silicone gel. In some examples, the silicone gel may be formed from a silicone fluid or from two or more silicone fluids having different viscosities. In some examples, the silicone gel may be produced by mixing a silicone fluid with a silicone crosslinked polymer. In some examples, the silicone gel may be produced by mixing a silicone fluid with a silicone gum. In some examples, the silicone gel may be produced by mixing a silicone fluid, a silicone crosslinked polymer and a silicone gum. The droplets are preferably dispersed in an aqueous phase using polymers that can provide a yield value. The size and shape of the silicone droplets are, in part, a function of the viscosity of the silicone gel and the yield value of the aqueous phase. The viscosity of the silicone gel may depend, in part, on the concentration of the gum or the crosslinked polymer.

Silicone fluids are generally linear polymers that have no cross-linking and, as such, are free to rotate. The viscosity of silicone fluids can vary considerably, but they are not solids. Suitable silicone fluids may include, but are not limited to, dimethicone (such as dimethicone having a viscosity of between about 0.65 cps and 100,000 cps), cyclomethicone, phenyl trimethicone, methyl trimethicone, amodimethicone, caprylyl methicone, disiloxane, trisiloxane, diphenyl dimethicone or methicone. Those skilled in the art will understand that other silicone fluids may be suitably used to produce a silicone gel or silicone droplets of the present disclosure.

At a certain level of viscosity, a silicone fluid may become too thick to pour, at which point it becomes a silicone gum. Suitable silicone gums may include, but are not limited to, dimethiconol. Those skilled in the art will understand that other silicone gums may be suitably used to produce a silicone gel or silicone droplets of the present disclosure.

As crosslinking is introduced between silicone polymers, their ability to freely rotate is compromised. Silicone crosslinked polymers typically exist in the form of an elastomer or a resin. Elastomers generally have a rubbery texture, whereas resins are generally hard or crumbly. The number and length of the crosslinking groups will typically determine if the resulting polymer is a resin or an elastomer. The silicone crosslinked polymers of the present disclosure are preferably elastomers. Suitable silicone crosslinked polymers may include dimethicone/vinyl dimethicone crosspolymer, dimethicone/phenyl vinyl dimethicone crosspolymer, vinyl dimethicone/lauryl dimethicone crosspolymer, divinyldimethicone/dimethicone/phenylsilsesquioxane crosspolymer, vinyldimethyl/trimethylsiloxysilicate stearyl dimethicone crosspolymer, dimethicone/vinyltrimethoxysiloxysilicate crosspolymer, vinyltrimethylsiloxysilicate/crosspolymer, polysilicone-11 or stearoxymethicone/dimethicone copolymer. Those skilled in the art will understand that other silicone crosslinked polymers may be suitably used to produce a silicone gel or silicone droplets of the present disclosure.

In certain examples, the silicone gel has a viscosity of between about 1 and 200,000 cps, or between about 10 and 150,000 cps, or between about 100 and 125,000 cps, or between about 500 and 110,000 cps, or between about 1,000 and 100,000 cps, or between about 1,250 and 90,000 cps, or between about 1,500 and 80,000 cps, or between about 1,750 and 70,000 cps, or between about 2,000 and 60,000 cps, or between about 2,000 and 50,000 cps, or between about 3,000 and 50,000 cps, or between about 4,000 and 50,000 cps, or between about 5,000 and 50,000 cps at 25° C.

In some examples, the composition of the present disclosure comprises silicone gel or silicone droplets at a concentration of between about 1% and 50% (w/w) such as between about 2% and 30% (w/w) or between about 10% and 25% (w/w). In some examples, the composition of the present disclosure comprises silicone gel or silicone droplets at a concentration of between about 2% and 20% (w/w), such as between about 5% and 17% (w/w), or between about 6% and 15% (w/w), or between about 7% and 14% (w/w), or between about 8% and 13% (w/w), or between about 9% and 12% (w/w), such as about 10% (w/w), or about 10.5% (w/w), or about 11% (w/w), or about 11.5% (w/w).

The silicone gel or silicone droplets of the present disclosure may be formed from: a silicone fluid; a silicone fluid and a silicone crosslinked polymer; a silicone fluid and a silicone gum; or a silicone fluid, a silicone crosslinked polymer and a silicone gum. In some examples, the silicone droplets also comprise other compounds such as film formers, emollients other than silicone, colourants, sunscreen agents, biologically active agents or cosmetically active agents.

The silicone gel and silicone droplets described herein may comprise a film former. A film former is typically a durable, water-resistant polymer which, upon drying, produces a continuous film. Examples of synthetic film forming polymers include, but are not limited to, those set forth in the CFTA Cosmetic Ingredient Dictionary and Handbook, Eight Edition, 2000, pages 1744 to 1747. In the compositions of the present disclosure, a film former may contribute to the formation of more uniform silicone droplets. Suitable film formers may comprise isodecane and trimethylsiloxysilylcarbamoyl pullulan (eg, TSPL-30-ID), isododecane (and) norbornene.tris(trimethylsiloxy)silyl norbornene copolymer, isododecane (and) acrylates/polytrimethylsiloxymethacrylate copolymer, acrylates/hydroxy esters acrylates copolymer, acrylates copolymer, dimethicone (and) trimethylsiloxysilicate, polyester-7, neopentyl glycol diheptanoate, butyl ester of PVM/MA copolymer, PVP, butylated PVP, VP/eicosene copolymer. Those skilled in the art will understand that other film formers may be used to produce a silicone gel or silicone droplets in accordance with the present disclosure. The film former may be present in the composition at a concentration of between about 0.05% and 15% (w/w), such as between about 0.1% and 14% (w/w), or between about 0.1% and 13% (w/w), or between about 0.1% and 12% (w/w), or between about 0.1% and 11% (w/w), or between about 0.1% and 10% (w/w), or between about 0.1% and 9% (w/w), or between about 0.15% and 8.5% (w/w), or between about 0.2% and 8% (w/w), or between about 0.2% and 5% (w/w), or between about 0.2% and 2.5% (w/w), or between about 0.2% and 1% (w/w), or between about 0.2% and 0.4% (w/w).

In some examples, the silicone gel and silicone droplets of the present disclosure further comprise an opacifier such as titanium dioxide, zinc oxide, mica (synthetic fluorphlogopite) and tin oxide and combinations thereof. Those skilled in the art will understand that other opacifiers may be suitably used in the compositions of the present disclosure.

The silicone gel and silicone droplets described herein may comprise a biologically active agent, a cosmetically active agent, an oil, an emollient other than silicone, a skin feel agent, a perfume, a skin whitening agent, a dye, a pigment, an antioxidant, a sun-screening agent or a soft focus powder. These additional ingredients may be added to the silicone gel from which the silicone droplets are formed.

Suitable antioxidants may include tocopherol, tocopheryl acetate, butylated hydroxytoluene (BHT), dimethylmethoxy chromanol, diethylhexyl syringylidenemalonate, octadecene dioic acid, ubiquinone (co-enzyme Q10) and rosemary leaf extract. Those skilled in the art will understand that other antioxidants may be suitably used in the compositions of the present disclosure.

Suitable oils may include natural oils from plants, for example, essential oils, seed oils or kernel oils, chemically-synthesized oils, for example, straight, branched or cyclic alkanes or alkenes, mineral oil, isododecane, isohexadecane, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, C12-15 alkyl benzoate, butyloctyl salicylate, phenylethyl benzoate, dicaprylyl carbonate, dioctyl maleate, dicaprylyl maleate, isononyl isononanoate, propylene glycol dicaprate, diisopropyl adipate, dibutyl adipate and oleyl adipate. Other suitable esters are described in the Personal Care Product Council's International Cosmetic Ingredient Dictionary and Handbook, under the functional category of “Esters.” Other suitable esters may include polyhydric alcohol esters and glycerides. Those skilled in the art will understand that other oils may be used to produce a silicone gel or silicone droplets in accordance with the present disclosure.

Suitable sun-screening agents may include octocylene, octinoxate, aminobenzoic acid, Parsol ZX (zinc oxide and triethoxycaprylylsilane), Parsol TX (titanium dioxide and silica and dimethicone), avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazolyl phenyl ethylhexyl imino triazine, drometrizole trisiloxane, methylene bis-benzotriazolyl tetramethylbutylphenol, bis-ethylhexyloxyphenol methoxyphenyl triazine, 4-methylbenzylidene camphor and isopentyl 4-methoxycinnarnate. Those skilled in the art will understand that other sunscreening agents may be used to produce a silicone gel or silicone droplets in accordance with the present disclosure. In examples where the silicone droplets comprise a sun-screening agent, the agent ray be present in the composition at a concentration of between about 0.5% and 10% (w/w) such as between about 1% and 7.5% (w/w), or between about 1% and 5% (w/w), or between about 1.5% and 3.5% (w/w), or between about 2% and 3% (w/w), or between about 2% and 2.5% (w/w).

Suitable oil-soluble skin whitening agents may include hydroxypinacolone retinoate, retinyl palmitate, undecylenoyl phenylalanine, ascorbyl palmitate and ascorbyl tetraisopalmitate. Those skilled in the art will understand that other oil-soluble skin whitening agents may be used to produce a silicone gel or silicone droplets in accordance with the present disclosure.

Suitable oil-soluble or oil-dispersible colourants may include natural or synthetic colourants or pigments. Those skilled in the art will understand that other oil-soluble and oil-dispersible colourants may be used to produce a silicone gel or silicone droplets in accordance with the present disclosure.

Suitable oil-soluble pigments may include inorganic pigments, metal oxides or hydroxides, mica, organic pigments, pearlescent pigments, mineral silicates, porous materials, carbons and interference pigments.

Suitable oil-soluble natural dyes may include Eclipta prostrata extract, Melia azadirachta leaf extract, Moringa pterygosperma oil (Campo® Siddha Vepuvillai Karushalai Yenai), Melia azadirachta seed extract (and) Melia azadirachta leaf extract (and) amber powder (and) Coccinia indica fruit extract (and) Solanum melongena (eggplant) fruit extract (and) Ocimum sanctum leaf extract (and) Ocimum basilicum (basil) flower/leaf extract (and) Curcuma longa (turmeric) leaf extract (and) Corallina officinalis extract (Campo Siddha Pazchai Thool), Melia azadirachta leaf extract (and) Melia azadirachta flower extract (and) amino esters-1 (ex Mimosa tenuiflora) (and) Coccinia indica fruit extract (and) Solanum melongena (eggplant) fruit extract (and) Aloe barbadensis flower extract (and) Simmondsia chinensis (Jojoba) seed oil (and) Ocimum sanctum leaf extract (and) Ocimum basilicum (basil) flower/leaf extract (and) Curcuma longa (turmeric) root extract (and) Corallina officinalis extract (Campo Siddha Indra Neelam Yenai) Melia azadirachta leaf extract (and) Melia azadirachta flower extract (and) amino esters-1 (ex Mimosa tenuiflora) (and) Coccinia indica fruit extract (and) Solanum melongena (eggplant) fruit extract (and) Aloe barbadensis flower extract (and) Simmondsia chinensis (Jojoba) seed oil (and) Ocimum sanctum leaf extract (and) Ocimum basilicum (basil) flower/leaf extract (and) Curcuma longa (turmeric) root extract (and) Corallina officinalis extract (Campo Siddha Indra Neelam Yenai).

In examples where the silicone gel or silicone droplets comprise a colourant, the colourant may be present in the composition at a concentration of between about 0.01% and 5% (w/w) such as between about 0.05% and 2.5% (w/w), or between about 0.05% and 1% (w/w), such as about 0.1% or about 0.2% (w/w).

The silicone droplets of the present disclosure may be clear, translucent, opaque, pearlised, coloured or colourless. Preferably, the silicone droplets are clear or translucent. The silicone droplets may be in the form of spheres, rods, strings, irregular shapes or combinations thereof. In some examples, two or more batches of silicone droplets can be prepared, each batch having a different visual appearance or textural properties. The batches of silicone droplets can then be combined to produce a single composition having a combination of desired visual or textural characteristics.

Suitable emollients other than silicone may include plant or algae extracts such as Caulerpa lentillifera oil or Rosa canina fruit extract. Those skilled in the art will understand that other emollients other than silicone may be used to produce a silicone gel or silicone droplets in accordance with the present disclosure.

Surprisingly, the inventor has found that visually and texturally pleasing droplet compositions that are able to carry lipophilic materials can be prepared in the absence of surfactants.

In some examples, the silicone droplets described herein may, in the absence of a surfactant, be used as a carrier for lipophilic compounds such as lipophilic emollients other than silicone or lipophilic active ingredients. The lipophilic compounds may be embedded in the silicone elastomer of a droplet and thereby separated from the aqueous phase and from other droplets in the composition. Lipophilic compounds that are susceptible to hydrolysis or are incompatible with surfactants may therefore be contained within the silicone droplets described herein. Suitable compounds that may be included in the silicone droplets of the present disclosure include vitamin A palmitate, hydroxypinacolone retinoate, ascorbyl palmitate, ascorbyl tetraisopalmitate, panthenyl triacetate and ethyl 3-(N-butylacetamido) propionate. Those skilled in the art will understand that other lipophilic compounds may be included in the silicone gel or silicone droplets of the present disclosure.

The compositions described herein may comprise two or more different compounds in a manner that avoids, or minimises, contact between the two or more compounds. In a sense, the compounds may be present in a single composition, but largely quarantined from each other. Such embodiments may be preferred where the two or more compounds are incompatible or reactive with each other.

In some examples, two batches of silicone droplets can be prepared, each batch comprising a different lipophilic compound embedded within the elastomeric matrix of the silicone droplets. The batches can then be combined to form a single composition comprising: an aqueous phase; a first dispersion of silicone droplets comprising a first lipophilic compound; and a second dispersion of silicone droplets comprising a second lipophilic compound. As each compound is contained within a separate dispersion of droplets, such compositions may include active compounds that are otherwise incompatible or reactive with each other.

In some examples, the present disclosure provides a composition comprising: at least one aqueous phase comprising water and a thickening agent; and at least one silicone gel, wherein the at least one silicone gel forms silicone droplets dispersed in the at least one aqueous phase when the composition is agitated. It will be understood, that each silicone gel may have different visual, textural, biological or cosmetic properties. For example, the composition may comprise an aqueous phase comprising water and a thickening agent; and two silicone gels each gel having a different respective colour, wherein the two silicone gels form silicone droplets dispersed in the aqueous phase when the composition is agitated. In such examples, the agitated composition will comprise two populations of dispersed silicone droplets distinguishable by their respective colour. In some examples, the present disclosure provides a composition comprising: at least one aqueous phase comprising water and a thickening agent; and two or more silicone gels, wherein the two or more silicone gels form silicone droplets dispersed in the at least one aqueous phase when the composition is agitated. In some examples, the present disclosure provides a composition comprising: at least one aqueous phase comprising water and a thickening agent; a first silicone gel comprising a first lipophilic compound; and a second silicone gel comprising a second lipophilic compound, wherein the first silicone gel and the second silicone gel form silicone droplets dispersed in the aqueous phase when the composition is agitated.

In certain examples, the aqueous phase of the composition may comprise a catalyst such as an enzyme and the silicone droplets may comprise a substrate for the catalyst. In such examples, the catalyst and its substrate may be brought into contact when the composition is topically applied to the skin, hair or nails of a subject. In that regard, the present disclosure provides a composition comprising silicone droplets dispersed in an aqueous phase wherein the aqueous phase comprises a catalyst and the silicone droplets comprise a substrate for the catalyst. In other examples, the catalyst and its substrate may be separately contained within respective silicone droplets, or the catalyst may be contained within the silicone droplets while the substrate is present in the aqueous phase.

In some examples, the present disclosure provides a composition comprising:

an aqueous phase comprising water and a thickening agent; and

silicone droplets dispersed in the aqueous phase, wherein the silicone droplets are formed from a silicone fluid.

In some examples, the present disclosure provides a composition comprising:

an aqueous phase comprising water and a thickening agent; and

silicone droplets dispersed in the aqueous phase, wherein the silicone droplets are formed from a silicone fluid and a silicone crosslinked polymer.

In some examples, the present disclosure provides a composition comprising:

an aqueous phase comprising water and a thickening agent; and

silicone droplets dispersed in the aqueous phase, wherein the silicone droplets are formed from a silicone fluid and optionally one or more of a silicone crosslinked polymer and a silicone gum,

wherein the composition comprises substantially no surfactant.

In some examples, the present disclosure provides a composition comprising:

an aqueous phase comprising water, a thickening agent, a humectant, a preservative and a chelating agent; and

silicone droplets dispersed in the aqueous phase, wherein the silicone droplets are formed from a silicone fluid and optionally one or more of a silicone crosslinked polymer and a silicone gum.

In some examples, the present disclosure provides a composition comprising:

an aqueous phase comprising water, a thickening agent, a humectant, a preservative and a chelating agent; and

silicone droplets dispersed in the aqueous phase, wherein the silicone droplets are formed from a silicone fluid and optionally one or more of a silicone crosslinked polymer and a silicone gum, wherein the composition comprises the silicone droplets in an amount of between about 9% and 25% (w/w).

In some examples, the present disclosure provides a composition comprising:

an aqueous phase comprising water, a thickening agent at a concentration in the composition of between about 0.6% and 1.2% (w/w), a humectant at a concentration in the composition of between about 4% and 14% (w/w), a preservative at a concentration in the composition of between about 0.1% and 1.5% (w/w), a chelating agent at a concentration in the composition of between about 0.05% and 0.5% (w/w); and

silicone droplets dispersed in the aqueous phase, wherein the silicone droplets are formed from a silicone fluid and optionally one or more of a silicone crosslinked polymer and a silicone gum, wherein the composition comprises the silicone droplets in an amount of between about 9% and 25% (w/w).

In some examples, the present disclosure provides a composition comprising:

an aqueous phase comprising water, a thickening agent and one or more of:
a humectant such as glycerine, propanediol, methyl propanediol or butylene glycol;
a preservative such as chlorphenesin, phenoxyethanol or benzyl alcohol;
a chelating agent such as tetrasodium EDTA, disodium EDTA or trisodium ethylene diamine disuccinate;
a skin lightening agent such as niacinamide or plankton extract;
an anti-irritant such as allantoin or alpha-bisabolol;
a perfuming agent such as Rosa damascena flower water; and
a colourant such as a dye (eg, FD&C Red No. 33 or FD&C Blue 1), a pigment, a mineral, a metal or a metal oxide; and

silicone droplets dispersed in the aqueous phase, wherein the silicone droplets are formed from a silicone fluid and optionally one or more of a silicone crosslinked polymer and a silicone gum, wherein the silicone droplets comprise one or more of:
an emollient other than silicone such as Caulerpa lentillifera oil or Rosa canina fruit extract;
a film former such as isododecane (and) trimethylsiloxysilyl-carbamoyl pullulan;
a colourant such as a pigment, a dye, a pearliser, mineral, a metal, a metal oxide or a plant extract;
a sun-screening agent such as octocrylene, octinoxate, diethylamino hydroxybenzoyl hexyl benzoate, zinc oxide and triethoxycaprylylsilane or titanium dioxide and silica; and
a lipophilic active agent such as a lipophilic cosmetically active agent or a lipophilic biologically active agent.

In some examples, the present disclosure provides a composition comprising:

an aqueous phase comprising water and a thickening agent; and

a silicone gel formed from a silicone fluid and optionally one or more of a silicone crosslinked polymer and a silicone gum,

wherein the silicone gel forms silicone droplets dispersed in the aqueous phase when the composition is agitated. Such compositions may be agitated by a subject who, for example, shakes the composition by hand to disperse the silicone gel, forming droplets, and then applies the composition topically, for example, to their skin or hair.

In some examples, the present disclosure provides a composition comprising:

an aqueous phase comprising water and a thickening agent; and

a silicone gel formed from a silicone fluid and optionally one or more of a silicone crosslinked polymer and a silicone gum,

wherein the silicone gel forms silicone droplets dispersed in the aqueous phase when the composition is agitated,

wherein the composition comprises substantially no surfactant.

In some examples, the present disclosure provides a composition comprising:

an aqueous phase comprising water and a thickening agent; and

a silicone gel formed from a silicone fluid and optionally one or more of a silicone crosslinked polymer and a silicone gum,

wherein the silicone gel forms silicone droplets dispersed in the aqueous phase when the composition is agitated,

wherein the composition comprises the silicone gel in an amount of between about 9% and 25% (w/w).

In some examples, the present disclosure provides a composition comprising:

an aqueous phase comprising water, a thickening agent at a concentration in the composition of between about 0.6% and 1.2% (w/w), a humectant at a concentration in the composition of between about 4% and 14% (w/w), a preservative at a concentration in the composition of between about 0.1% and 1.5% (w/w), a chelating agent at a concentration in the composition of between about 0.05% and 0.5% (w/w); and

a silicone gel formed from a silicone fluid and optionally one or more of a silicone crosslinked polymer and a silicone gum,

wherein the silicone gel forms silicone droplets dispersed in the aqueous phase when the composition is agitated,

wherein the composition comprises the silicone gel in an amount of between about 9% and 25% (w/w).

In some examples, the present disclosure provides a composition comprising:

an aqueous phase comprising water, a thickening agent and one or more of:
a humectant such as glycerine, propanediol, methyl propanediol or butylene glycol;
a preservative such as chlorphenesin, phenoxyethanol or benzyl alcohol;
a chelating agent such as tetrasodium EDTA, disodium EDTA or trisodium ethylene diamine disuccinate;
a skin lightening agent such as niacinamide or plankton extract;
an anti-irritant such as allantoin or alpha-bisabolol;
a perfuming agent such as Rosa damascena flower water; and
a colourant such as a dye (eg, FD&C Red No. 33 or FD&C Blue 1), a pigment, a mineral, a metal or a metal oxide; and

a silicone gel formed from a silicone fluid and optionally one or more of a silicone crosslinked polymer and a silicone gum, wherein the silicone gel comprises one or more of:
an emollient other than silicone such as Caulerpa lentillifera oil or Rosa canina fruit extract;
a film former such as isododecane (and) trimethylsiloxysilyl-carbamoyl pullulan;
a colourant such as a pigment, a dye, a pearliser, mineral, a metal, a metal oxide or a plant extract;
a sun-screening agent such as octocrylene, octinoxate, diethylamino hydroxybenzoyl hexyl benzoate, zinc oxide and triethoxycaprylylsilane or titanium dioxide and silica; and
a lipophilic active agent such as a lipophilic cosmetically active agent or a lipophilic biologically active agent,

wherein the silicone gel forms silicone droplets dispersed in the aqueous phase when the composition is agitated.

In some examples, the present disclosure provides a composition comprising:

an aqueous phase comprising:
water;
a thickening agent selected from the group consisting of polyacrylate crosspolymer-6, Xanthan gum, Caesalpinia spinosa gum, Sclerotium gum and sodium polyacrylate starch;
a humectant selected from the group consisting of propylene glycol, glycine, butylene glycol, propanediol and methyl propanediol;
a chelating agent selected from the group consisting of trisodium ethylene diamine disuccinate, tetrasodium EDTA and disodium EDTA; and
a preservative selected from the group consisting of phenoxyethanol, benzyl alcohol and chlorphenesin; and

silicone droplets dispersed in the aqueous phase, wherein the silicone droplets are formed from:
a silicone fluid;
a silicone fluid and a silicone crosslinked polymer;
a silicone fluid and a silicone gum; or
a silicone fluid, a silicone crosslinked polymer and a silicone gum.

In some examples, the present disclosure provides a composition comprising:

an aqueous phase comprising water, butylene glycol, propanediol, xanthan gum, sclerotium gum, phenoxyethanol, chlorphenesin and disodium EDTA; and

silicone droplets dispersed in the aqueous phase, wherein the silicone droplets are formed from dimethicone/vinyl dimethicone crosspolmer and cyclopentasiloxane.

In some examples, the present disclosure provides a composition comprising:

an aqueous phase comprising: water; methyl propanediol; propanediol; sodium polyacrylate starch; phenoxyethanol; chlorphenesin; and disodium EDTA; and

silicone droplets dispersed in the aqueous phase, wherein the silicone droplets are formed from: dimethicone; dimethicone and dimethicone/vinyl dimethicone crosspolymer; and a film former.

In some examples, the present disclosure provides a kit comprising: an aqueous phase comprising water and a thickening agent; and a silicone gel, wherein the silicone gel forms silicone droplets dispersed in the aqueous phase when the silicone gel is mixed with the aqueous phase to form a composition and the composition is agitated. In some examples, the present disclosure provides a kit comprising: an aqueous phase comprising water and a thickening agent; a silicone gel; and instructions for using the kit by mixing the aqueous phase with the silicone gel to form a composition and agitating the composition so as to form silicone droplets dispersed in the aqueous phase.

As described herein, the compositions of the present invention are stable under various conditions. For example, the compositions of the present disclosure preferably maintain their visual appearance, viscosity and pH for extended periods at different temperatures. In some examples, the present disclosure provides a composition that is stable at 50° C. for at least 1 week, or at least 2 weeks, or at least 3 weeks, or at least 1 month, or at least 2 months. In some examples, the present disclosure provides a composition that is stable at 25° C. for at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 12 months, or at least 13 months, or at least 14 months, or at least 15 months, or at least 16 months, or at least 17 months, or at least 18 months, or at least 19 months, or at least 20 months, or at least 21 months, or at least 22 months, or at least 23 months, or at least 24 months, or at least 25 months, or at least 26 months, or at least 27 months, or at least 28 months, or at least 29 months, or at least 30 months, or at least 31 months, or at least 32 months, or at least 33 months, or at least 34 months, or at least 35 months, or at least 36 months.

The composition may be in the form of a gel, a cream, a face wash, a body wash, a foundation, a paste, a cleansing product, a lotion, a moisturiser, a shampoo, a conditioner, a scalp maintenance product, a hair styling product, a serum, a toner, a hydrogel, a gel cream, a solution, a water cream, a gel to cream, a cream to gel, a cream to powder, a foundation, a shaving product or a skin-lightening product. Those skilled in the art will understand that the compositions described herein may take many other forms. Preferably, the composition is a cosmetic composition.

### Methods and Uses

The present disclosure provides methods for in situ dispersion of a silicone gel in an aqueous phase to form silicone droplets. In some examples, the present disclosure provides a method of producing a composition comprising silicone droplets dispersed in an aqueous phase, the method comprising:

stirring an aqueous phase comprising water and a thickening agent; and

adding a silicone gel to the aqueous phase as it is being stirred so as to form silicone droplets in the aqueous phase.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid and optionally one or more of a silicone crosslinked polymer and a silicone gum to thereby form a silicone gel;

c) stirring the aqueous phase; and

d) adding the silicone gel to the aqueous phase as it is being stirred so as to form silicone droplets dispersed in the aqueous phase.

It will be understood that steps a) and b) may be performed in any order, or simultaneously, but are performed separately, for example, in separate vessels.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid and one or more of a silicone crosslinked polymer and a silicone gum to thereby form a silicone gel;

c) stirring the aqueous phase; and

d) adding the silicone gel to the aqueous phase as it is being stirred so as to form silicone droplets dispersed in the aqueous phase,

wherein the composition comprises substantially no surfactant.

The stirring may be performed at a rate of between about 50 rpm and 1000 rpm, such as between about 60 rpm and 750 rpm, or between about 75 rpm and 700 rpm, or between about 100 rpm and 650 rpm, or between about 110 rpm and 600 rpm, or between about 115 rpm and 550 rpm, or between about 125 rpm and 500 rpm, or between about 150 rpm and 450 rpm. To produce large droplets, slower stirring speeds should be used. To produce small droplets, faster stirring speeds should be used. Suitable stirrers may include, but are not limited to, a paddle, a propeller blade stirrer or a magnetic stirrer.

The silicone droplets may be round and may have a diameter of between about 0.01 mm and 10 mm, such as between about 0.1 mm and 5 mm, or between about 0.2 mm and 4 mm, or between about 0.3 mm and 3.5 mm, or between about 0.4 mm and 3 mm, or between about 0.5 mm and 3 mm, or between about 1 mm and 3 mm, or between about 1.5 mm and 3 mm, or between about 1.5 mm and 2.5 mm.

It will be understood that the aqueous phase, and the combination of the aqueous phase and the silicone gel, may be agitated using a technique other than stirring. For example, the aqueous phase, and the combination of the aqueous phase and the silicone gel, may be vibrated, sonicated, shaken or otherwise exposed to shear forces.

In some examples, the present disclosure provides a method of producing a composition comprising silicone droplets dispersed in an aqueous phase, the method comprising:

a) mixing water, a thickening agent, a humectant, a preservative and a chelating agent to thereby produce an aqueous phase;

b) mixing a silicone fluid and a silicone crosslinked polymer to thereby form a silicone gel;

c) stirring the aqueous phase; and

d) adding the silicone gel to the aqueous phase as it is being stirred so as to form silicone droplets dispersed in the aqueous phase.

In some examples, the present disclosure provides a method of producing a composition comprising silicone droplets dispersed in an aqueous phase, the method comprising:

adding a silicone gel to an aqueous phase, wherein the silicone gel is formed from a silicone fluid and optionally one or more of a silicone crosslinked polymer and a silicone gum, and wherein the aqueous phase comprises water and a thickening agent; and

agitating the silicone gel and the aqueous phase such that the silicone gel forms silicone droplets dispersed in the aqueous phase.

In some examples, the present disclosure provides a method of producing a composition comprising silicone droplets dispersed in an aqueous phase, the method comprising:

adding a silicone gel to an aqueous phase, wherein the silicone gel is formed from a silicone fluid and optionally one or more of a silicone crosslinked polymer and a silicone gum, and wherein the aqueous phase comprises water and a thickening agent; and

agitating the silicone gel and the aqueous phase such that the silicone gel forms silicone droplets dispersed in the aqueous phase,

wherein the composition comprises substantially no surfactant.

In some examples, the present disclosure provides a method of producing a composition comprising silicone droplets dispersed in an aqueous phase, the method comprising:

a) mixing: water; a thickening agent selected from the group consisting of polyacrylate crosspolymer-6, Xanthan gum, Caesalpinia spinosa gum, Sclerotium gum and sodium polyacrylate starch; a humectant selected from the group consisting of propylene glycol, glycine, butylene glycol, propanediol and methyl propanediol; a preservative selected from the group consisting of phenoxyethanol, benzyl alcohol and chlorphenesin; and a chelating agent selected from the group consisting of trisodium ethylene diamine disuccinate, tetrasodium EDTA and disodium EDTA to thereby produce an aqueous phase;

b) mixing: a silicone fluid and a silicone crosslinked polymer; a silicone fluid and a silicone gum; or a silicone fluid, a silicone crosslinked polymer and a silicone gum to thereby form a silicone gel;

c) stirring the aqueous phase; and

d) adding the silicone gel to the aqueous phase as it is being stirred so as to form silicone droplets dispersed in the aqueous phase.

In some examples, the present disclosure provides a method of producing a composition comprising silicone droplets dispersed in an aqueous phase, the method comprising:

a) mixing water, butylene glycol, propanediol, xanthan gum, sclerotium gum, phenoxyethanol, chlorphenesin and disodium EDTA to thereby produce an aqueous phase;

b) mixing a film former and dimethicone/vinyl dimethicone crosspolmer and cyclopentasiloxane to thereby form a silicone gel;

c) stirring the aqueous phase; and

d) adding the silicone gel to the aqueous phase as it is being stirred so as to form silicone droplets dispersed in the aqueous phase.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid and one or more of a silicone crosslinked polymer and a silicone gum to thereby produce a silicone gel;

c) adding the silicone gel to the aqueous phase; and

d) agitating the silicone gel and the aqueous phase such that the silicone gel forms silicone droplets dispersed in the aqueous phase.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid and one or more of a silicone crosslinked polymer and a silicone gum to thereby produce a silicone gel;

c) adding the silicone gel to the aqueous phase; and

d) agitating the silicone gel and the aqueous phase such that the silicone gel forms silicone droplets dispersed in the aqueous phase, wherein the composition comprises substantially no surfactant.

In some examples, the present disclosure provides a method of producing a composition comprising silicone droplets dispersed in an aqueous phase, the method comprising:

a) mixing water, methyl propanediol, propanediol, sodium polyacrylate starch, phenoxyethanol, chlorphenesin and disodium EDTA to thereby produce an aqueous phase;

b) mixing dimethicone, dimethicone and dimethicone/vinyl dimethicone crosspolymer, and a film former to thereby form a silicone gel;

c) stirring the aqueous phase; and

d) adding the silicone gel to the aqueous phase as it is being stirred so as to form silicone droplets dispersed in the aqueous phase.

The silicone droplets described herein may further comprise a biologically or cosmetically active agent, an emollient other than silicone, a dye, a pigment, a sun-screening agent or a soft focus powder. In some examples, the silicone droplets described herein may be used as a carrier for lipophilic compounds such as lipophilic emollients other than silicone or lipophilic active agents. These additional ingredients may be included in the silicone gel from which the silicone droplets are formed.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid, a silicone crosslinked polymer and a compound selected from the group consisting of a biologically active agent, a cosmetically active agent, an emollient other than silicone, a dye, a pigment, a sun-blocking agent, a lipophilic active agent and a soft focus powder to thereby form a silicone gel;

c) stirring the aqueous phase; and

d) adding the silicone gel to the aqueous phase as it is being stirred so as to form silicone droplets dispersed in the aqueous phase.

In some examples, the present disclosure provides a method of producing a composition comprising silicone droplets dispersed in an aqueous phase, the method comprising:

a) mixing water, a thickening agent and one or more of:
a humectant such as glycerine, propanediol, methyl propanediol or butylene glycol;
a preservative such as chlorphenesin, phenoxyethanol or benzyl alcohol;
a chelating agent such as tetrasodium EDTA, disodium EDTA or trisodium ethylene diamine disuccinate;
a skin lightening agent such as niacinamide or plankton extract;
an anti-irritant such as allantoin or alpha-bisabolol;
a perfuming agent such as Rosa damascena flower water; and
a colourant such as a dye (eg, FD&C Red No. 33 or FD&C Blue 1), a pigment, a mineral, a metal or a metal oxide,

to thereby form an aqueous phase;

b) mixing a silicone fluid, optionally one or more of a silicone crosslinked polymer and a silicone gum, and one or more of:
an emollient other than silicone such as Caulerpa lentillifera oil or Rosa canina fruit extract;
a film former such as isododecane (and) trimethylsiloxysilyl-carbamoyl pullulan;
a colourant such as a pigment, a dye, a pearliser, mineral, a metal, a metal oxide or a plant extract;
a sun-screening agent such as octocrylene, octinoxate, diethylamino hydroxybenzoyl hexyl benzoate, zinc oxide and triethoxycaprylylsilane or titanium dioxide and silica; and
a lipophilic active agent such as a lipophilic cosmetically active agent or a lipophilic biologically active agent,

to thereby form a silicone gel;

c) adding the silicone gel to the aqueous phase; and

d) agitating the silicone gel and the aqueous phase such that the silicone gel forms silicone droplets dispersed in the aqueous phase.

In some examples, the present disclosure provides a method of producing a composition comprising silicone droplets dispersed in an aqueous phase, the method comprising:

a) mixing water and a thickening agent to thereby produce an aqueous phase;

b) mixing a silicone fluid, a silicone crosslinked polymer and a lipophilic compound to thereby form a silicone gel;

c) stirring the aqueous phase; and

d) adding the silicone gel to the aqueous phase as it is being stirred so as to form silicone droplets dispersed in the aqueous phase.

In some examples, the present disclosure provides a method of producing a composition comprising silicone droplets dispersed in an aqueous phase the method comprising:

mixing water with a silicone gel, wherein the silicone gel comprises a thickening agent; and

agitating the silicone gel and the water such that at least some of the thickening agent is dissolved in the water and the silicone gel forms silicone droplets dispersed in the aqueous phase.

### EXAMPLES

### Example 1

An aqueous phase was prepared by mixing the compounds listed in Table 1. The concentration of each compound in the final composition (ie, after the addition of the silicone gel) is indicated in the table.

TABLE 1
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Methylpropanediol | Dub Diol | Humectant | 10.00 |
| Xanthan gum | Vanzan NFC | Thickening | 0.30 |
| | | agent | |
| Sclerotium gum | Amigum | Thickening | 0.70 |
| | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating | 0.10 |
| | | agent | |
| Cl 42090 and Aqua | FD&C Blue 1, | Colourant | 0.005 |
| | 1% Aqueous | | |
| | Solution | | |
| Plankton extract, | Brighlette | Skin | 2.00 |
| butylene glycol, and | | lightening | |
| water | | agent | |
| Glyceryl ascorbate, | Amitose 3GA | Antioxidant | 2.00 |
| glycerine and water | | | |

In a separate vessel, a silicone gel was prepared by mixing the compounds listed in Table 2.

TABLE 2
Silicone gel

| | | | Concentration in |
|---|---|---|---|
| Ingredient | Trade | | final composition |
| (INCI Name) | name | Function | (w/w %) |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |
| Isododecane (and) | TSPL-30 | Film former | 0.20 |
| trimethylsiloxysilyl- | ID | | |
| carbamoyl pullulan | | | |

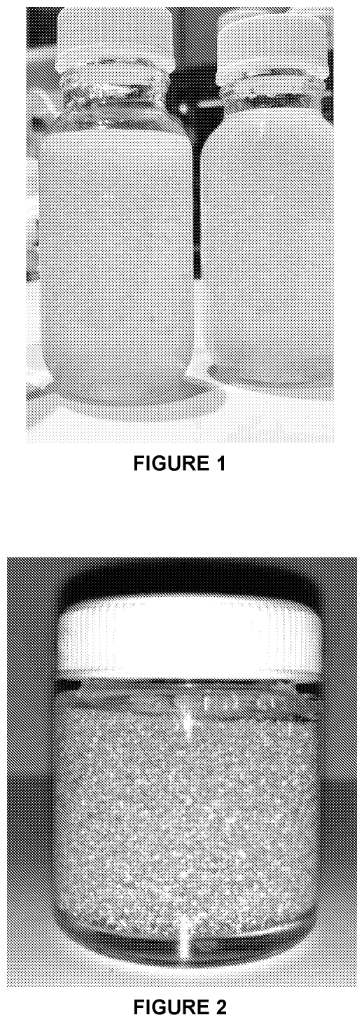

The silicone gel was slowly added to the aqueous phase as the aqueous phase was being stirred, resulting in the formation of dispersed silicone droplets (FIG. 1).

The stability of the composition was tested at various temperatures. The results of the stability testing is set forth in Table 3.

TABLE 3
Stability data

| | Initial | | 6 months | |
|---|---|---|---|---|
| Physical | (Unaged | 1 month | (40 C./75% | 20 months |
| Property | control) | 50 C. | Humidity) | (25 C.) |
| Appearance | Translucent | Comparable | Comparable | Comparable |
| | serum with | to unaged | to unaged | to unaged |
| | dispersed | control | control | control |
| | transparent | | | |
| | droplets | | | |
| Viscosity @ | 6,000 cps | 6,400 cps | 6,200 cps | 6,800 cps |
| 25 C. | | | | |
| (RVT, | | | | |
| Spindle #TD, | | | | |
| 10 rpm) | | | | |
| pH @ 25 C. | 3.51 | 3.49 | 3.49 | 3.48 |

### Example 2

An aqueous phase was prepared by mixing the compounds listed in Table 4.

TABLE 4
Aqueous phase

| | | | Concentration |
|---|---|---|---|
| Ingredient | | | in final |
| | | | composition |
| (INCI Name) | Trade name | Function | (w/w %) |
| Aqua (Purified Water) | | | q.s. |
| Tetrasodium EDTA | EDETA B | Chelating | 0.10 |
| | | agent | |
| Chlorphenesin | Elestab CPN | Preservative | 0.30 |
| Allantoin | Allantoin | Anti-irritant | 0.10 |
| Niacinamide | Niacinamide PC | Skin | 4.00 |
| | | lightening | |
| | | agent | |
| Glycerine | Glycerine | Humectant | 4.00 |
| Propanediol | Zemea | Humectant | 6.00 |
| Polyacrylate | Sepimax Zen | Thickening | 0.60 |
| crosspolymer-6 | | agent | |
| Xanthan gum | Vanzan NFC | Thickening | 0.10 |
| | | agent | |
| Phenoxyethanol | Phenoxyethanol | Preservative | 0.50 |
| Cl 42090 and Water | 1% FD&C | Colourant | 0.01 |
| | Blue 1 aqueous | | |
| | solution | | |
| Melia Azadirachta | Campo Siddha | Colourant | 0.15 |
| Flower Extract, Melia | Ellam Neer | | |
| Azadirachta Leaf | Manjal | | |
| Extract, Lawsonia | | | |
| Inermis (Henna) | | | |
| Flower/Fruit/Leaf | | | |
| Extract, Ocimum | | | |
| Sanctum Leaf Extract, | | | |
| Ocimum Basilicum | | | |
| (Basil) Flower/Leaf | | | |
| Extract, Curcuma | | | |
| Longa (Turmeric) Root | | | |
| Extract, Mica, Pearl | | | |
| Powder (Margarita | | | |
| Powder), Aqua | | | |

In a separate vessel, an opaque silicone gel was prepared as set out in Table 5.

TABLE 5
Silicone gel

| | | | Concentration |
|---|---|---|---|
| | | | in final |
| Ingredient | | | composition |
| (INCI Name) | Trade name | Function | (w/w %) |
| Dimethicone | Xiameter 200, | Silicone fluid | 1.00 |
| | 200 cst | | |
| Cyclomethicone | Xiameter 345 | Silicone fluid | 2.00 |
| | Fluid | | |
| Dimethicone and | Serasilk PD 73 | Silicone fluid | 8.00 |
| dimethicone/vinyl | | and Silicone | |
| dimethicone | | cross-linked | |
| crosspolymer | | polymer | |
| | | (Elastomer) | |
| Titanium dioxide | Parsol TX | Opacifier | 0.20 |
| and Silica and | | | |
| Dimethicone | | | |

The silicone gel was slowly added to the aqueous phase while the aqueous phase was being stirred, resulting in the formation of dispersed silicone droplets (FIG. 2).

### Example 3

An aqueous phase was prepared by mixing the compounds listed in Table 6.

TABLE 6
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Rosa damascena | Rose Water | Perfuming | 40.00 |
| flower water | | | |
| Disodium EDTA | Versene NA2 | Chelating | 0.20 |
| | | agent | |
| Allantoin | Allantion | Anti-irritant | 0.20 |
| Trehalose | Trehalose | Humectant | 0.25 |
| Butylene glycol | Butylene Glycol | Humectant | 4.00 |
| Propanediol | Zemea | Humectant | 6.00 |
| | Propanediol | | |
| Sodium polyacrylate | Makimousse 7 | Thickening | 0.60 |
| starch | | agent | |
| Chlorphenesin | Elestab CPN | Preservative | 0.30 |
| Phenoxyethanol | Phenoxyethanol | Preservative | 0.50 |
| Cl 17200 and Aqua | FD&C Red | Colourant | 0.01 |
| | #33, 1% | | |
| | aqueous | | |
| | solution | | |

In a separate vessel, a pearlised silicone gel was prepared by mixing the compounds listed in Table 7.

TABLE 7
Silicone gel

| | | | Concentration |
|---|---|---|---|
| | | | in final |
| Ingredient | | | composition |
| (INCI Name) | Trade name | Function | (w/w %) |
| Isododecane (and) | TSPL-30-ID | Film former | 0.40 |
| trimethylsiloxysilyl- | | | |
| carbamoyl pullulan | | | |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |
| Alumina (and) titanium | Timiron | Pearliser | 0.20 |
| dioxide (and) tin oxide | Glam Silver | pigment | |

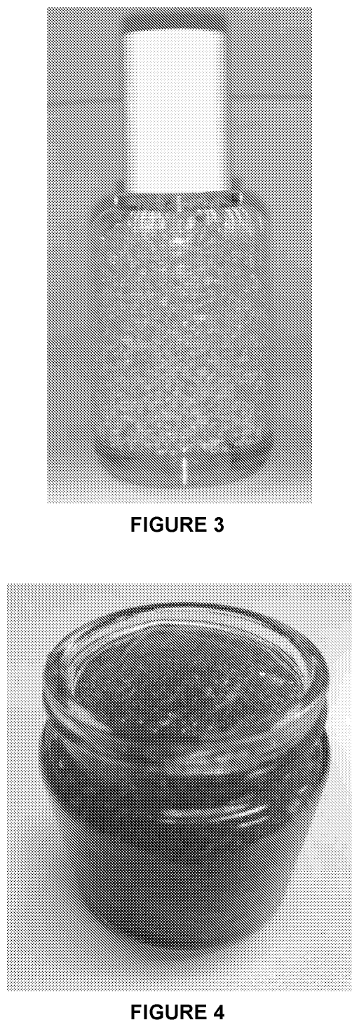

The silicone gel was slowly added to the aqueous phase as the aqueous phase was being stirred, resulting in the formation of dispersed silicone droplets (FIG. 3).

The stability of the composition was tested at various temperatures. The results of the stability testing is set forth in Table 8.

TABLE 8
Stability data

| | Initial | | 6 months | |
|---|---|---|---|---|
| Physical | (Unaged | 1 month | (40 C./75% | 20 months |
| Property | control) | 50 C. | Humidity) | (25 C.) |
| Appearance | Translucent | Comparable | Comparable | Comparable |
| | pink Serum | to unaged | to unaged | to unaged |
| | with dispersed | control | control | control |
| | pearly droplets | | | |
| Viscosity @ | 4,200 cps | 4,200 cps | 4,300 cps | 4,500 cps |
| 25 C. | | | | |
| (RVT, | | | | |
| Spindle #TD, | | | | |
| 10 rpm) | | | | |
| pH @ 25 C. | 6.98 | 7.01 | 6.95 | 6.93 |

### Example 4

An aqueous phase was prepared by mixing the compounds listed in Table 9.

TABLE 9
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Disodium EDTA | Versene NA2 | Chelating | 0.10 |
| | | agent | |
| Propanediol | Zemea | Humectant | 10.00 |
| | Propanediol | | |
| Sodium polyacrylate | Makimousse 25 | Thickening | 0.35 |
| starch | | agent | |
| Chlorphenesin | Elestab CPN | Preservative | 0.30 |
| Phenoxyethanol | Phenoxyethanol | Preservative | 0.50 |
| Melia Azadirachta Leaf | Campo Siddha | Colourant | 0.15 |
| Extract, Melia | Neer Karum | | |
| Azadirachta Flower | Karuppu | | |
| Extract, Amino-Esters- | | | |
| 1, Eclipta Prostrata | | | |
| Extract, Lawsonia | | | |
| Inermis (Henna) | | | |
| Flower/Fruit/Leaf | | | |
| Extract, Ocimum | | | |
| Sanctum Leaf Extract, | | | |
| Corallina Officinalis | | | |
| Extract, Aqua. | | | |

In a separate vessel, a sparkling silicone gel was prepared by mixing the compounds listed in Table 10.

TABLE 10
Silicone gel

| | | | Concentration |
|---|---|---|---|
| | | | in final |
| Ingredient | Trade | | composition |
| (INCI Name) | name | Function | (w/w %) |
| Dimethicone and | Dowsil 3901 | Silicone fluid | 0.40 |
| Dimethicone/Vinyl | | and Silicone | |
| Dimethicone | | cross-linked | |
| Crosspolymer | | polymer | |
| | | (Elastomer) | |
| Dimethicone and | Gransil | Silicone fluids | 8.00 |
| Cyclopentasiloxane | DMCM-5 | and Silicone | |
| and Polysilicone-11 | | cross-linked | |
| | | polymer | |
| | | (Elastomer) | |
| Dimethicone | Xiameter 200, | Silicone fluid | 2.00 |
| | 1000 cs | | |
| Mica and Titanium | Timiron | Sparkling | 0.20 |
| Dioxide | Snowflake | agent | |
| | MP-99 | | |

The silicone gel was slowly added to the aqueous phase as the aqueous phase was being stirred, resulting in the formation of dispersed silicone droplets in the form of rods (FIG. 4).

### Example 5

An aqueous phase was prepared by mixing the compounds listed in Table 11.

TABLE 11
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Disodium EDTA | Versene NA2 | Chelating agent | 0.10 |
| Propanediol | Zemea | Humectant | 10.00 |
| | Propanediol | | |
| Sodium polyacrylate | Makimousse 25 | Thickening | 0.35 |
| starch | | agent | |
| Chlorphenesin | Elestab CPN | Preservative | 0.30 |
| Phenoxyethanol | Phenoxyethanol | Preservative | 0.50 |
| Melia Azadirachta | Campo | Colourant | 0.15 |
| Flower Extract, Melia | Siddha Neer | | |
| Azadirachta Leaf | Ellam Manjal | | |
| Extract, Lawsonia | | | |
| Inermis (Henna) | | | |
| Flower/Fruit/Leaf | | | |
| Extract, Ocimum | | | |
| Sanctum Leaf Extract, | | | |
| Ocimum Basilicum | | | |
| (Basil) Flower/Leaf | | | |
| Extract, Curcuma | | | |
| Longa (Turmeric) Root | | | |
| Extract, Mica, Pearl | | | |
| Powder (Margarita | | | |
| Powder), Aqua | | | |
| Aqua, Melia | Campo | Colourant | 0.15 |
| Azadirachta Leaf | Siddha Neer | | |
| Extract, Melia | Ellam Gopi | | |
| Azadirachta Flower | | | |
| Extract, Amino-Esters- | | | |
| 1, Betaine, Coccinia | | | |
| Indica Fruit Extract, | | | |
| Pearl Powder | | | |
| (Margarita Powder) | | | |

In a separate vessel, a pearlised silicone gel was prepared by mixing the compounds listed in Table 12.

TABLE 12
Silicone gel

| | | | Concentration |
|---|---|---|---|
| | | | in final |
| Ingredient | Trade | | composition |
| (INCI Name) | name | Function | (w/w %) |
| Dimethicone and | Dowsil 3901 | Silicone fluid | 0.50 |
| Dimethicone/Vinyl | | and Silicone | |
| Dimethicone | | cross-linked | |
| Crosspolymer | | polymer | |
| | | (Elastomer) | |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |
| Mica and Titanium | Timiron Supersheen | Pearlising | 0.20 |
| Dioxide | MP-1001 | Pigment | |

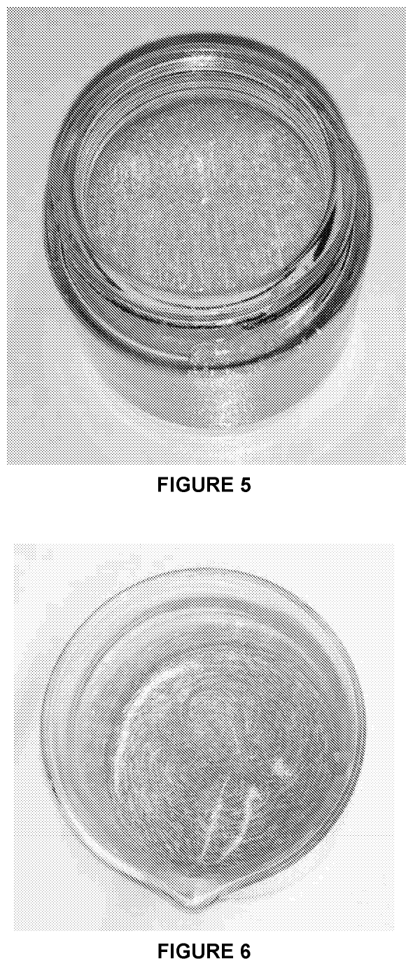

The silicone gel was slowly added to the aqueous phase as the aqueous phase was being stirred, resulting in the formation of dispersed silicone droplets in the form of strings (FIG. 5).

### Example 6

An aqueous phase was prepared by mixing the compounds listed in Table 13.

TABLE 13
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Disodium EDTA | Versene NA2 | Chelating | 0.10 |
| | | agent | |
| Propanediol | Zemea | Humectant | 10.00 |
| | Propanediol | | |
| Sodium polyacrylate | Makimousse 25 | Thickening | 0.35 |
| starch | | agent | |
| Chlorphenesin | Elestab CPN | Preservative | 0.30 |
| Phenoxyethanol | Phenoxyethanol | Preservative | 0.50 |
| Melia Azadirachta | Campo | Colourant | 0.15 |
| Flower Extract, Melia | Siddha Neer | | |
| Azadirachta Leaf | Ellam Manjal | | |
| Extract, Lawsonia | | | |
| Inermis (Henna) | | | |
| Flower/Fruit/Leaf | | | |
| Extract, Ocimum | | | |
| Sanctum | | | |
| Leaf Extract, | | | |
| Ocimum Basilicum | | | |
| (Basil) Flower/ | | | |
| Leaf Extract | | | |
| Curcuma Longa | | | |
| (Turmeric) Root | | | |
| Extract, Mica, Pearl | | | |
| Powder (Margarita | | | |
| Powder), Aqua | | | |
| Aqua, Melia | Campo | Colourant | 0.15 |
| Azadirachta Leaf | Siddha Neer | | |
| Extract, Melia | Ellam Gopi | | |
| Azadirachta Flower | | | |
| Extract, Amino- | | | |
| Esters-1, | | | |
| Betaine, Coccinia | | | |
| Indica Fruit Extract, | | | |
| Pearl Powder | | | |
| (Margarita Powder) | | | |

In a separate vessel, a pearly silicone gel was prepared by mixing the compounds listed in Table 14.

TABLE 14
Silicone gel

| | | | Concentration |
|---|---|---|---|
| | | | in final |
| Ingredient | Trade | | composition |
| (INCI Name) | name | Function | (w/w %) |
| Dimethicone and | Dowsil 3901 | Silicone fluid | 10.00 |
| Dimethicone/Vinyl | | and Silicone | |
| Dimethicone | | cross-linked | |
| Crosspolymer | | polymer | |
| | | (Elastomer) | |
| Alumina (and) titanium | Timiron Glam | Pearliser | 0.20 |
| dioxide (and) tin oxide | Silver | pigment | |

The silicone gel was slowly added to the aqueous phase as the aqueous phase was being stirred, resulting in the formation of dispersed silicone droplets in the form of strings (FIG. 6).

The stability of the composition was tested and the results are set forth in Table 15.

TABLE 15
Stability data

| | Initial | 1 month |
|---|---|---|
| Physical Property | (Unaged control) | 50 C. |
| Appearance | Translucent orange | Comparable to unaged |
| | gel with dispersed | control |
| | pearly strings | |
| Viscosity @ 25 C. | 12,000 cps | 12,600 cps |
| (RVT, Spindle #TD, | | |
| 10 rpm) | | |
| pH @ 25 C. | 7.30 | 7.35 |

### Example 7

An aqueous phase was prepared by mixing the compounds listed in Table 16.

TABLE 16
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Methylpropanediol | Dub Diol | Humectant | 6.00 |
| Glycerine | Glycerine | Humectant | 4.00 |
| Xanthan gum | Vanzan NFC | Thickening | 0.30 |
| | | agent | |
| Sclerotium gum | Amigum | Thickening | 0.70 |
| | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating agent | 0.10 |
| Glyceryl Ascorbate, | Amitose 3GA | Antioxidant | 2.00 |
| and Glycerine and | | | |
| Aqua | | | |
| Plankton Extract and | Brighlette | Skin Whitening | 2.00 |
| Butylene Glycol and | | Active | |
| Water | | | |

In a separate vessel, a coloured (red) silicone gel was prepared by mixing the compounds listed in Table 17.

TABLE 17
Silicone gel

| | | | Concentration |
|---|---|---|---|
| | | | in final |
| Ingredient | Trade | | composition |
| (INCI Name) | name | Function | (w/w %) |
| Dimethicone/vinyl | KSG-15 | Silicone cross- | 5.00 |
| dimethicone | | linked polymer | |
| crosspolmer and | | (Elastomer) and | |
| cyclopentasiloxane | | Silicone fluid | |
| Isododecane (and) | TSPL-30 ID | Film former | 0.19 |
| trimethylsiloxysilyl- | | | |
| carbamoyl pullulan | | | |
| Fragrance | Perfume Chantiq | Perfuming | 0.10 |
| Eclipta Prostrata | Campo ® Siddha | Colourant | 0.03 |
| Extract, Melia | Vepuvillai | | |
| Azadirachta Leaf | Karushalai | | |
| Extract, Moringa | Yenai | | |
| Pterygosperma Oil | | | |
| Dimethicone and | Dowsil 9041 | Silicone fluid | 5.00 |
| Dimethicone | | and Silicone | |
| Crosspolymer | | cross-linked | |
| | | polymer | |
| | | (Elastomer) | |

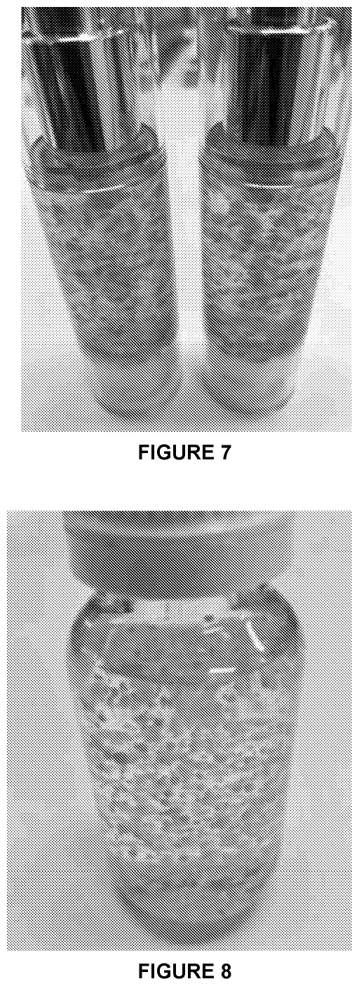

The silicone gel was slowly added to the aqueous phase as the aqueous phase was being stirred, resulting in the formation of dispersed silicone droplets (FIG. 7).

The stability of the composition was tested and the results are set forth in Table 18.

TABLE 18
Stability data

| Physical | Initial | 1 month |
|---|---|---|
| Property | (Unaged control) | 50 C. |
| Appearance | Translucent gel | Comparable to unaged |
| | with dispersed red | control |
| | droplets | |
| Viscosity @ 25 C. | 7,000 cps | 7,100 cps |
| (RVT, Spindle | | |
| #TD, 10 rpm) | | |
| pH @ 25 C. | 3.60 | 3.58 |

### Example 8

An aqueous phase was prepared by mixing the compounds listed in Table 19.

TABLE 19
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Butylene glycol | Butylene Glycol | Humectant | 5.00 |
| Propanediol | Zemea | Humectant | 5.00 |
| Sodium polyacrylate | Makimousse 7 | Thickening | 0.70 |
| starch | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating agent | 0.1 |
| Niacinamide | Niacinamide PC | Skin lightening | 4.00 |
| | | agent | |
| Alpha-bisabolol | Bisabolol, Nat. | Anti-irritant | 0.50 |

In a separate vessel, a silicone gel comprising UV filters was prepared by mixing the compounds listed in Table 20.

TABLE 20
Silicone gel

| | | | Concentration |
|---|---|---|---|
| | | | in final |
| Ingredient | Trade | | composition |
| (INCI Name) | name | Function | (w/w %) |
| Caulerpa lentillifera oil | Green Caviar Oil | Emollient | 2.00 |
| Caprylyl methicone | Silsoft 034 | Silicone fluid | 1.00 |
| Zinc oxide and | Parsol ZX | UV Filter | 1.00 |
| triethoxycaprylylsilane | | | |
| Titanium dioxide and | Parsol TX | UV Filter | 1.00 |
| silica and dimethicone | | | |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |

The silicone gel was slowly added to the aqueous phase as the aqueous phase was being stirred, resulting in the formation of dispersed silicone droplets (FIG. 8).

### Example 9

An aqueous phase was prepared by mixing the compounds listed in Table 21.

TABLE 21
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Glycerine | Glycerine | Humectant | 4.00 |
| Propanediol | Zemea | Humectant | 6.00 |
| Sodium polyacrylate | Makimousse 7 | Thickening | 0.70 |
| starch | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating agent | 0.1 |
| Niacinamide | Niacinamide PC | Skin lightening | 4.00 |
| | | agent | |
| Allantoin | Allantoin | Anti-irritant | 0.10 |

In a separate vessel, a silicone gel comprising UV filters was prepared by mixing the compounds listed in Table 22.

TABLE 22
Silicone gel

| | | | Concentration |
|---|---|---|---|
| | | | in final |
| Ingredient | Trade | | composition |
| (INCI Name) | name | Function | (w/w %) |
| Octocrylene | Parsol 340 | UVB sunscreen | 0.50 |
| Octinoxate | Parsol MCX | UVB sunscreen | 1.5 |
| Diethylamino | Uvinul A Plus | UVA sunscreen | 0.25 |
| hydroxybenzoyl hexyl | | | |
| benzoate | | | |
| Caprylyl methicone | Silsoft 034 | Silicone fluid | 1.00 |
| Dimethicone/vinyl | KSG-15 | Silicone cross- | 20.00 |
| dimethicone | | linked polymer | |
| crosspolmer and | | (Elastomer) and | |
| cyclopentasiloxane | | Silicone fluid | |
| Isododecane (and) | TSPL-30-ID | Film former | 0.40 |
| trimethylsiloxysilyl- | | | |
| carbamoyl pullulan | | | |

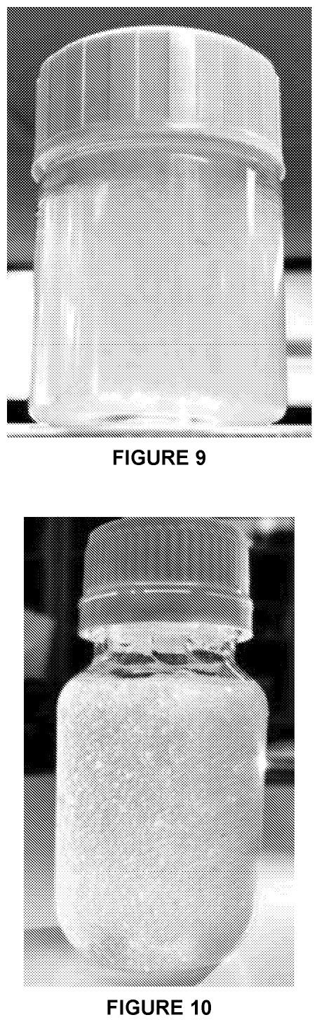

The silicone gel was slowly added to the aqueous phase as the aqueous phase was being stirred, resulting in the formation of dispersed silicone droplets (FIG. 9).

### Example 10

An aqueous phase was prepared by mixing the compounds listed in Table 23.

TABLE 23
Aqueous phase

| | | | Concentration |
|---|---|---|---|
| | | | in final |
| Ingredient | Trade | | composition |
| (INCI Name) | name | Function | (w/w %) |
| Aqua (Purified Water) | | | q.s. |
| Methylpropanediol | Dub Diol | Humectant | 10.00 |
| Xanthan gum | Vanzan NFC | Thickening agent | 0.30 |
| Sclerotium gum | Amigum | Thickening agent | 0.70 |
| Phenoxyethanol | Phenoxyethanol | Preservative | 0.50 |
| Chlorphenesin | Elestab CPN | Preservative | 0.30 |
| Disodium EDTA | Versene NA2 | Chelating agent | 0.10 |

In a separate vessel, a silicone gel was prepared by mixing the compounds listed in Table 24.

TABLE 24
Silicone gel

| | | | Concentration |
|---|---|---|---|
| | | | in final |
| Ingredient | Trade | | composition |
| (INCI Name) | name | Function | (w/w %) |
| Dimethicone/vinyl | KSG-15 | Silicone cross-linked | 10.00 |
| dimethicone | | polymer (Elastomer) | |
| crosspolmer and | | and Silicone fluid | |
| cyclopentasiloxane | | | |
| Isododecane (and) | TSPL-30 | Film former | 0.20 |
| trimethylsiloxysilyl- | ID | | |
| carbamoyl pullulan | | | |
| Hydroxypinacolone | Granactive | Active | 1.00 |
| retinoate | Retinoid | | |

The silicone gel was slowly added to the aqueous phase as the aqueous phase was being stirred, resulting in the formation of dispersed, transparent silicone droplets (FIG. 10).

### Example 11

An aqueous phase was prepared by mixing the compounds listed in Table 25.

TABLE 25
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Glycerine | Glycerine | Humectant | 4.00 |
| Propanediol | Zemea | Humectant | 6.00 |
| Sodium polyacrylate | Makimousse 7 | Thickening | 0.70 |
| starch | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating | 0.1 |
| | | agent | |
| Niacinamide | Niacinamide PC | Skin | 4.00 |
| | | lightening | |
| | | agent | |
| Alpha-bisabolol | Bisabolol, Nat. | Anti-irritant | 0.50 |

In a separate vessel, a coloured silicone gel was prepared by mixing the compounds listed in Table 26.

TABLE 26
Silicone gel

| | | | Concentration |
|---|---|---|---|
| | | | in final |
| Ingredient | | | composition |
| (INCI Name) | Trade name | Function | (w/w %) |
| Caulerpa lentillifera oil | Green Caviar | Emollient | 2.00 |
| | Oil | | |
| Caprylyl methicone | Silsoft 034 | Silicone fluid | 1.00 |
| Melia Azadirachta Seed | Campo | Natural | 0.10 |
| Extract (and) Melia | Siddha | colourant | |
| Azadirachta Leaf | Pazchai | | |
| Extract (and) Amber | Thool | | |
| Powder (and) Coccinia | | | |
| Indica Fruit Extract | | | |
| (and) Solanum | | | |
| Melongena (Eggplant) | | | |
| Fruit Extract (and) | | | |
| Ocimum Sanctum Leaf | | | |
| Extract (and) Ocimum | | | |
| Basilicum (Basil) | | | |
| Flower/Leaf Extract | | | |
| (and) Curcuma Longa | | | |
| (Turmeric) Leaf Extract | | | |
| (and) Corallina | | | |
| Officinalis Extract | | | |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |

The silicone gel was added to a bottle as one layer, followed by the aqueous phase as a second layer. Shaking the composition resulted in dispersion of the silicone gel as droplets in the aqueous phase (FIG. 11).

### Example 12

An aqueous phase was prepared by mixing the compounds listed in Table 27.

TABLE 27
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Rosa damascena | Rose Water | Perfuming | 40.00 |
| flower water | | | |
| Disodium EDTA | Versene NA2 | Chelating | 0.10 |
| | | agent | |
| Butylene glycol | Butylene Glycol | Humectant | 4.00 |
| Glycerine | Glycerine | Humectant | 6.00 |
| Sodium polyacrylate | Makimousse 7 | Thickening | 0.60 |
| starch | | agent | |
| Chlorphenesin | Elestab CPN | Preservative | 0.30 |
| Phenoxyethanol | Phenoxyethanol | Preservative | 0.50 |
| Allantion | Allantoin | Anti-irritant | 0.20 |
| Trehalose | Trehalose | Humectant | 0.25 |
| Saccharide Isomerate | Pentavitin | Moisturiser | 1.00 |
| and Aqua and Citric | | | |
| Acid and Sodium | | | |
| Citrate | | | |
| Cl 17200 and Aqua | FD&C Red # | Colourant | 0.01 |
| | 33, 1% aqueous | | |
| | solution | | |

In a separate vessel, a clear silicone gel was prepared by mixing the compounds listed in Table 28.

TABLE 28
Silicone gel

| | | | Concentration in |
|---|---|---|---|
| Ingredient | | | final composition |
| (INCI Name) | Trade name | Function | (w/w %) |
| Dimethicone/vinyl | KSG-15 | Silicone cross- | 10.00 |
| dimethicone | | linked polymer | |
| crosspolmer and | | (Elastomer) | |
| cyclopentasiloxane | | and Silicone | |
| | | fluid | |
| Isododecane (and) | TSPL-30 ID | Film former | 0.19 |
| trimethylsiloxysilyl- | | | |
| carbamoyl pullulan | | | |

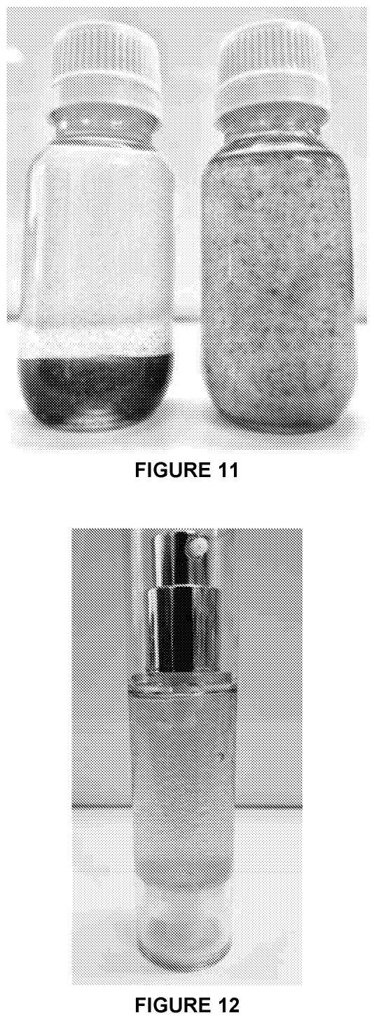

The silicone gel was slowly added to the aqueous phase as the aqueous phase was being stirred, resulting in the formation of dispersed, clear silicone droplets (FIG. 12).

The stability of the composition was tested and the results are set forth in Table 29.

TABLE 29
Stability data

| Physical | Initial | 1 month |
|---|---|---|
| Property | (Unaged control) | 50 C. |
| Appearance | Clear droplets | Comparable to unaged |
| | dispersed in | control |
| | aqueous phase | |
| Viscosity @ 25 C. | 6,000 cps | 6,200 cps |
| (RVT, Spindle | | |
| #TD, 10 rpm) | | |
| pH @ 25 C. | 7.00 | 7.03 |

### Example 13

An aqueous phase was prepared by mixing the compounds listed in Table 30.

TABLE 30
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Glycerine | Glycerine | Humectant | 10.00 |
| Sodium polyacrylate | Makimousse 25 | Thickening | 0.35 |
| starch | | agent | |
| Benzyl alcohol | Benzyl Alcohol | Preservative | 0.50 |
| Chlorphenesin | Elestab CPN | Preservative | 0.30 |
| Tetrasodium EDTA | EDETA B | Chelating | 0.10 |
| | | agent | |

In a separate vessel, a silicone gel was prepared by mixing the compounds listed in Table 31.

TABLE 31
Silicone gel

| | | | Concentration |
|---|---|---|---|
| | | | in final |
| Ingredient | | | composition |
| (INCI Name) | Trade name | Function | (w/w %) |
| Dimethicone | Xiameter 200, | Silicone | 7.00 |
| | 10000 cst Fluid | fluid | |
| Dimethicone and | Xiameter PMX- | Silicone | 3.00 |
| Dimethiconol | 1403 Fluid | fluid and | |
| | | silicone | |
| | | gum | |
| Melia Azadirachta Leaf | Campo Siddha | Colourant | 0.10 |
| Extract (and) Melia | Indra Neelam | | |
| Azadirachta Flower | Yenai | | |
| Extract (and) Amino | | | |
| Esters-1 (ex Mimosa | | | |
| Tenuiflora) (and) Coccinia | | | |
| Indica Fruit Extract (and) | | | |
| Solanum Melongena | | | |
| (Eggplant) Fruit Extract | | | |
| (and) Aloe Barbadensis | | | |
| Flower Extract (and) | | | |
| Simmondsia Chinensis | | | |
| (Jojoba) Seed Oil (and) | | | |
| Ocimum Sanctum Leaf | | | |
| Extract (and) Ocimum | | | |
| Basilicum (Basil) | | | |
| Flower/Leaf Extract (and) | | | |
| Curcuma Longa | | | |
| (Turmeric) Root Extract | | | |
| (and) Corallina Officinalis | | | |
| Extract | | | |

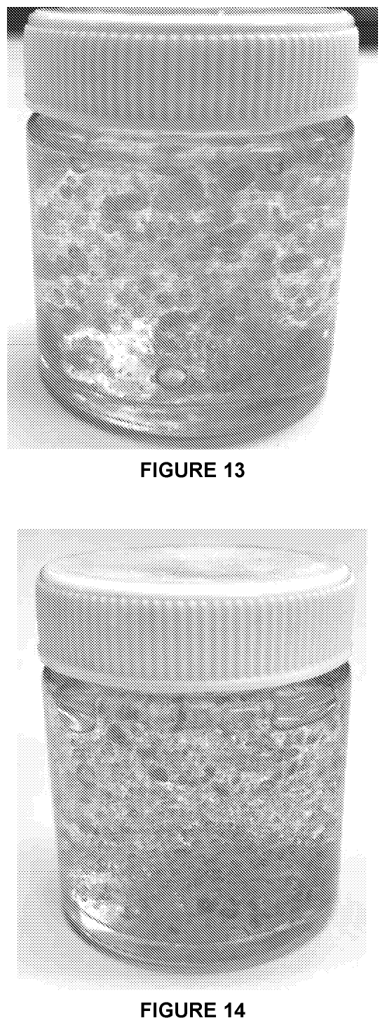

The silicone gel was slowly added to the aqueous phase as the aqueous phase was being stirred, resulting in the formation of dispersed silicone droplets (FIG. 13).

### Example 14

An aqueous phase was prepared by mixing the compounds listed in Table 32.

TABLE 32
Aqueous phase

| | | | Concentration |
|---|---|---|---|
| | | | in final |
| Ingredient | | | composition |
| (INCI Name) | Trade name | Function | (w/w %) |
| Aqua (Purified | | | q.s. |
| Water) | | | |
| Glycerine | Glycerine | Humectant | 10.00 |
| Xanthan gum | Vanzan NFC | Thickening | 0.20 |
| | | agent | |
| Sclerotium gum | Amigum | Thickening | 0.80 |
| | | agent | |
| Phenoxyethanol | Phenoxyethanol | Preservative | 0.50 |
| Sodium Benzoate | Sodium Benzoate | Preservative | 0.50 |
| Disodium EDTA | Versene NA2 | Chelating agent | 0.10 |

In a separate vessel, a silicone gel was prepared by mixing the compounds listed in Table 33.

TABLE 33
Silicone gel

| | | | Concentration |
|---|---|---|---|
| | | | in final |
| Ingredient | | | composition |
| (INCI Name) | Trade name | Function | (w/w %) |
| Dimethicone | Xiameter 200, | Silicone fluid | 7.00 |
| | 60000 cst | | |
| Eclipta prostrata extract | Campo Siddha | Colourant | 0.10 |
| and Melia azadirachta | Villai | | |
| leaf extract and Moringa | Karushalai | | |
| pterygosperma oil | Yenai | | |

The silicone gel was slowly added to the aqueous phase as the aqueous phase was being stirred, resulting in the formation of dispersed silicone droplets (FIG. 14).

### Example 15

A composition comprising two layers was produced by first preparing two separate compositions. A first layer (bottom layer) of aqueous phase was prepared by mixing the compounds listed in Table 34.

TABLE 34
First aqueous phase

| | | | Concentration |
|---|---|---|---|
| | | | in final |
| Ingredient | | | composition |
| (INCI Name) | Trade name | Function | (w/w %) |
| Aqua (Purified | | | q.s. |
| Water) | | | |
| Glycerine | Glycerine | Humectant | 10.00 |
| Xanthan gum | Vanzan NFC | Thickening | 0.20 |
| | | agent | |
| Sclerotium gum | Amigum | Thickening | 0.80 |
| | | agent | |
| Phenoxyethanol | Phenoxyethanol | Preservative | 0.50 |
| Sodium Benzoate | Sodium Benzoate | Preservative | 0.50 |
| Disodium EDTA | Versene NA2 | Chelating agent | 0.10 |

In a separate vessel, a gold coloured silicone gel was prepared by mixing the compounds listed in Table 35.

TABLE 35
First silicone gel

| | | | Concentration |
|---|---|---|---|
| | | | in final |
| Ingredient | | | composition |
| (INCI Name) | Trade name | Function | (w/w %) |
| Dimethicone/vinyl | KSG-15 | Silicone cross- | 10.00 |
| dimethicone | | linked polymer | |
| crosspolmer and | | (Elastomer) | |
| cyclopentasiloxane | | and Silicone | |
| | | fluid | |
| Isododecane (and) | TSPL-30 ID | Film former | 0.19 |
| trimethylsiloxysilyl- | | | |
| carbamoyl pullulan | | | |
| Mica and Titanium | Candurin Gold | Pearlising | 0.20 |
| dioxide and Ferric | Sparkle | agent | |
| Oxide | | | |

The first silicone gel was added to the first aqueous phase as the first aqueous phase was being stirred, resulting in the formation of a first composition comprising gold coloured silicone droplets dispersed in an aqueous phase.

A second layer (top layer) of aqueous phase was prepared by mixing the compounds listed in Table 36.

TABLE 36
Second aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Butylene glycol | Butylene Glycol | Humectant | 5.00 |
| Propanediol | Zemea | Humectant | 5.00 |
| Sodium polyacrylate | Makimousse 7 | Thickening | 0.70 |
| starch | | agent | |
| Phenoxyethanol | Phenoxyethanol | Preservative | 0.50 |
| Chlorphenesin | Elestab CPN | Preservative | 0.30 |
| Disodium EDTA | Versene NA2 | Chelating | 0.1 |
| | | agent | |
| Niacinamide | Niacinamide PC | Skin | 4.00 |
| | | lightening | |
| | | agent | |

In a separate vessel, a second silicone gel comprising titanium dioxide was prepared by mixing the compounds listed in Table 37.

TABLE 37
Second silicone gel

| | | | Concentration |
|---|---|---|---|
| | | | in final |
| Ingredient | | | composition |
| (INCI Name) | Trade name | Function | (w/w %) |
| Titanium dioxide and | Parsol TX | UV Filter | 1.00 |
| silica and dimethicone | | | |
| Dimethicone/vinyl | KSG-15 | Silicone cross- | 10.00 |
| dimethicone | | linked polymer | |
| crosspolmer and | | (Elastomer) and | |
| cyclopentasiloxane | | Silicone fluid | |
| Isododecane (and) | TSPL-30 ID | Film former | 0.19 |
| trimethylsiloxysilyl- | | | |
| carbamoyl pullulan | | | |

The second silicone gel was slowly added to the second aqueous phase as the second aqueous phase was being stirred, resulting in the formation of a second composition comprising silicone droplets dispersed in an aqueous phase.

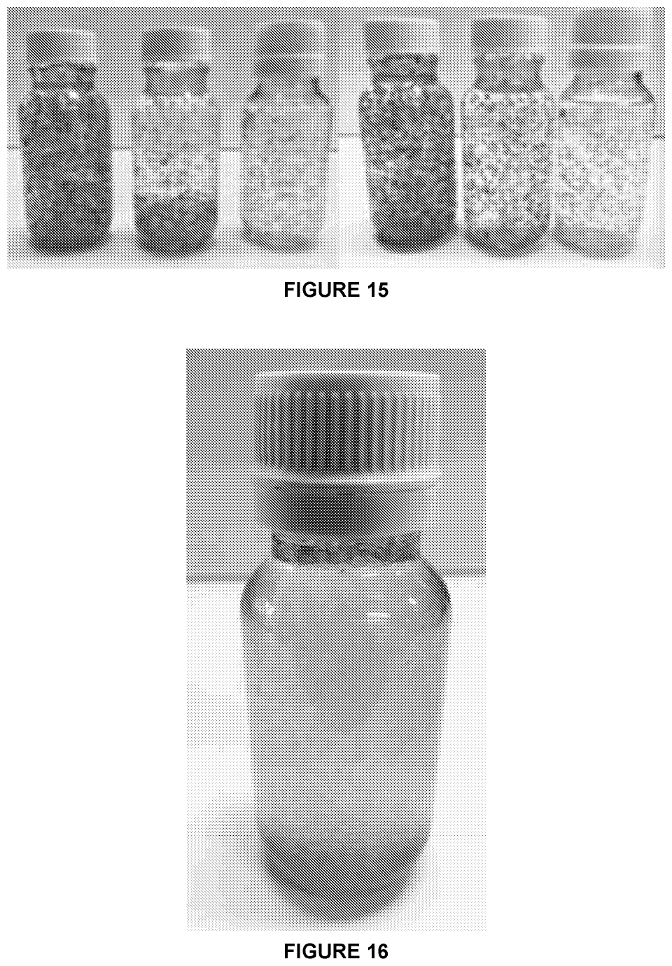

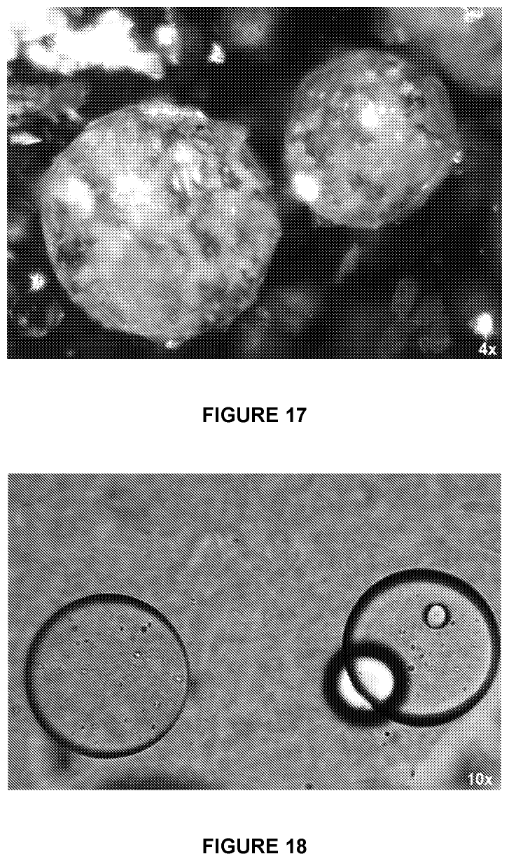

The first composition and second composition were combined in a bottle. The combined composition was agitated in order to mix the first and second compositions (FIG. 15).

### Example 16

An aqueous phase was prepared by mixing the compounds listed in Table 38.

TABLE 38
Aqueous phase

| | | | Concentration |
|---|---|---|---|
| | | | in final |
| Ingredient | | | composition |
| (INCI Name) | Trade name | Function | (w/w %) |
| Aqua (Purified Water) | | | q.s. |
| Rosa damascena | Rose Water | Perfuming | 40.00 |
| flower water | | | |
| Disodium EDTA | Versene NA2 | Chelating | 0.10 |
| | | agent | |
| Butylene glycol | Butylene Glycol | Humectant | 4.00 |
| Glycerine | Glycerine | Humectant | 6.00 |
| Sodium polyacrylate | Makimousse 7 | Thickening | 0.60 |
| starch | | agent | |
| Chlorphenesin | Elestab CPN | Preservative | 0.30 |
| Phenoxyethanol | Phenoxyethanol | Preservative | 0.50 |
| Allantion | Allantoin | Anti-irritant | 0.20 |
| Trehalose | Trehalose | Humectant | 0.25 |
| Saccharide Isomerate | Pentavitin | Moisturiser | 1.00 |
| and Aqua and Citric | | | |
| Acid and Sodium | | | |
| Citrate | | | |
| Cl 17200 and Aqua | FD&C Red # | Colourant | 0.01 |
| | 33, 1% aqueous | | |
| | solution | | |

In a separate vessel, a silicone gel was prepared by mixing the compounds listed in Table 39.

TABLE 39
Silicone gel

| | | | Concentration |
|---|---|---|---|
| | | | in final |
| Ingredient | | | composition |
| (INCI Name) | Trade name | Function | (w/w %) |
| Cyclopentasiloxane | Dowsil 9045 | Silicone fluid | 12.00 |
| and Dimethicone | | and Silicone | |
| Crosspolymer | | cross-linked | |
| | | polymer | |
| | | (Elastomer) | |
| Dimethicone | Xiameter200, 100 cst | Silicone fluid | 1.50 |
| Lauroyl lysine | Amihope LL | Texture | 0.05 |
| | | Modifier and | |
| | | Translucency | |
| | | agent | |

The silicone gel was slowly added to the aqueous phase as the aqueous phase was being stirred, resulting in the formation of dispersed, translucent silicone droplets (FIG. 16).

It will be appreciated by those skilled in the art that the compositions and methods of the present disclosure may be embodied in many other forms.

1-54. (canceled)

55. A composition comprising silicone droplets dispersed in an aqueous phase.

56. The composition of claim 55, wherein the composition comprises substantially no surfactant.

57. A cosmetic composition comprising:
an aqueous phase comprising water and a thickening agent; and
a silicone gel,
wherein the silicone gel forms silicone droplets dispersed in the aqueous phase when the composition is agitated.

58. The composition of claim 57, wherein the composition comprises substantially no surfactant.

59. The composition of claim 57, wherein the silicone gel is formed from a silicone fluid.

60. The composition of claim 57, wherein the silicone gel is formed from a silicone fluid and a silicone crosslinked polymer.

61. The composition of claim 57, wherein the silicone gel is formed from a silicone fluid and a silicone gum.

62. The composition of claim 57, wherein the silicone gel is formed from a silicone fluid, a silicone crosslinked polymer and a silicone gum.

63. The composition of claim 57, wherein the thickening agent is a gelling agent.

64. The composition of claim 57, wherein the aqueous phase further comprises a humectant.

65. The composition of claim 57, wherein the aqueous phase further comprises a preservative.

66. The composition of claim 57, wherein the aqueous phase further comprises a colourant.

67. The composition of claim 57, wherein the silicone gel comprises dimethicone/vinyl dimethicone crosspolymer.

68. The composition of claim 57, wherein the silicone gel comprises dimethicone.

69. The composition of claim 57, wherein the silicone gel comprises a film former.

70. The composition of claim 57, wherein the silicone gel comprises a lipophilic emollient other than silicone.

71. The composition of claim 57, wherein the silicone gel comprises a lipophilic active agent.

72. The composition of claim 57, wherein the composition comprises the silicone gel at a concentration of between about 1% and 50% (w/w).

73. The composition of claim 57, wherein the silicone gel forms silicone droplets dispersed in the aqueous phase when the composition is shaken or stirred.

74. A method of producing a composition comprising silicone droplets dispersed in an aqueous phase, the method comprising:
stirring an aqueous phase comprising water and a thickening agent; and
adding a silicone gel to the aqueous phase as it is being stirred so as to form silicone droplets dispersed in the aqueous phase.
